SEQUENCE LISTING (55 sequences)

SEQ ID NO: 1 - DNA, 351 bp, Panicum virgatum
  misc_feature (1)..(351): Ceres Gemini ID NO 9001H5
gcaccaacttcgtctacacgcacgccgcctacaactaccccccgttcctggcgccgttccacgcgcagccgtcgtcgtacgcgcacgcgccgtcgtccgtgcagtacggcggcgcgggcgcgccgcacattggctcgtaccaccaccaccaccaccactaccaggcttcggcggcggggtccggcggcgccctcctcgtcgtcggggggagtgctcggtgccggtggccgtggatcgcgccngacggcacgctgctgatggaccgcaacgggcacgancttcctgttcgcgagcgcggacgacaactccgggtacctgagcagcgtggtcccggagagctgcctccggccg

SEQ ID NO: 2 - DNA, 375 bp, Panicum virgatum
  misc_feature (1)..(375): Ceres Gemini ID NO 9001A5
ccttcgcggtcaagaacatctccgccgacaccttcgtcgccgacgccgcctccgtcccgccctccggcttctgggccccgagctccctcctcccccgcctctcctccctggacccccgcgccggcatggccttcgcctctggaaggtaaatcccgaagcggcgcacatgtgctcacaatttgtccactcgatcatacacatgggcacacaacatgatcgaactgatcgcatgcaagttcaactgctggtggttgctgcaggttctactgcatgagctcgtcgccgttcgcggtgctcgtgttcgacgtggcggccaacgagtggagcaaggtgcagccgccgatgaggaggttcctgcggtcgccggcgctcgtg

SEQ ID NO: 3 - DNA, 527 bp, Panicum virgatum
  misc_feature (1)..(527): Ceres Gemini ID NO 9001B5
cggggcagccggcgatgaacatgatgggagcaccgtcgacaagcgaatatgatcacatggcccctacgactcgagaaacttccttcaagtgaatattatgcagcagcctcagcactactcccatcagaggagggagagcaacagctgcagcaggtgaccgtggcccggtctgccgccatggccgccgccagtgcggagctgaacccattcttggagatggacaccaagtgcttcttccccgccggccccttcgcggggctggacatgaagtgcttctttccaggaggcttgcagatgctggaggcacaccgccagatactcaccaccgagctcaacctcggctaccaactggggttgatgagaatgaaagggcacagcagacagcgaacatgatgggggagtcgtcgacgagtgagtaccagcaaggtttcattccttatgacccaataagaagcttcctgcagttcaacatcacgcagcagcagcctcaattttactcccagcaggaggaccggaaagacttcaac

SEQ ID NO: 4 - DNA, 1383 bp, Arabidopsis thaliana
  misc_feature: Ceres Clone ID no. 123905
caaaaacacaaacaaaactcatattttcaatctccaggtgctttacaccaacagagtcgcaagaaaacaaaaaccaaactcggatttagtttgacagaagaaggaatcgagagtcgggtatgcattatcctaacaacagaaccgaattcgtcggagctccagccccaacccggtatcaaaaggagcagttgtcaccggagcaagagctttcagttattgtctctgctttgcaacacgtgatctcaggggaaaacgaaacggcgccgtgtcagggtttttccagtgacagcacagtgataagcgcgggaatgcctcggttggattcagacacttgtcaagtctgtaggatcgaaggatgtctcggctgtaactactttttcgcgccaaatcagagaattgaaaagaatcatcaacaagaagaagagattactagtagtagtaacagaagaagagagagctctcccgtggcgaagaaagcggaaggtggcgggaaaatcaggaagaggaagaacaagaagaatggttacagaggagttaggcaaagaccttggggaaaatttgcagctgagatcagagatcctaaaagagccacacgtgtttggcttggtactttcgaaaccgccgaagatgcggctcgagcttatgatcgagccgcgattggattccgtgggccaagggctaaactcaacttcccctttgtggattacacgtcttcagtttcatctcctgttgctgctgatgatataggagcaaatgcaagtgcaagcgccagtgtgagcgccacagattcagttgaagcagagcaatggaacggaggaggaggggattgcaatatggagtggatgaatatgatgatgatgatggattttgggaatggagattcttcagattcaggaaatacaattgctgatatgttccagtgataaatgagctctttcttgttggcgttttttggagttaagtgcaagaagagattgacactgtggcttgtttaaagtgaacaagaacaagaaagcatgtaattagtagtctcattcttttgtttgtggtcaattctatgtttatctcatataaaatctgagttaaacctatctgaggagagagtaaataaagaggttaagaaacccaacattggtctgaattataaacgtaagtgtcaacgttgtttataaaggagaaaactataattggtgacaaaagacataaagaaaagatgtctactcctacaaagcatcgcgtgcagctattcgacaaacaatggcatctcccagagaggaaattccgagctcttggctagttatcttgtaatgctgaaaacatgaatgtatttgagtttatttctgtaacattggaagcgaaataaaagggttatcaactgttacc

SEQ ID NO: 5 - protein, 268 aa, Arabidopsis thaliana
  misc_feature: Ceres Clone ID no. 123905
MHYPNNRTEFVGAPAPTRYQKEQLSPEQELSVIVSALQHVISGENETAPCQGFSSDSTVISAGMPRLDSDTCQVCRIEGCLGCNYFFAPNQRIEKNHQQEEEITSSSNRRRESSPVAKKAEGGGKIRKRKNKKNGYRGVRQRPWGKFAAEIRDPKRATRVWLGTFETAEDAARAYDRAAIGFRGPRAKLNFPFVDYTSSVSSPVAADDIGAKASASASVSATDSVEAEQWNGGGGDCNMEEWMNMMMMMDFGNGDSSDSGNTIADMFQ

SEQ ID NO: 6 - protein, 215 aa, Vitis vinifera
  misc_feature: GI ID no. 47852612
MRMFGDGMKIVESTAWPGLNKDVEFAVMVSTLQNVITGNIEPLQNDTFTTHHSNDLTVLALPDPEKCQECGFDGCLGCNFFAPPDERGKKRTRKRKYRGVRHRPWGKWAAEIRDPQKAVRLWLGTFDNAEAAARAYDRKAIEFRGIKAKLNFPLSDYTNETESSNIMGVRVKPTTSDLGESRKLKSKEKLHDAVDESELSKKKTAVAGEASRVRE

SEQ ID NO: 7 - DNA, 1023 bp, Zea mays
  misc_feature: Ceres Clone ID no. 1494990
aaacaaccatcaccttcaagcttagctccagcctccagccatcactcagctcaaggcacaatcaggcactcatcggcagcaagaacacaccgaccttcagcgtctcggcgtcaatggaggcgagccggcagtacatgatccgcttcgacggccacttcgaggagggcccgagctccgcggccgccgagccaccgcagccgttcgccagcagggctttctcgccggagcaggagcagagcgtcatggtcgccgcgctgctgcacgtcgtctccgggtacgccacgccggcgccggacctcttcttcccggcgggcaaggaggcgtgcacggcgtgcggggtggacgggtgcctcggctgcgagttcttcggcgccgaggccgggcgcgcggtcgcggcatcggacgcgccgagagcggcgaccgctggcgggccgcagaggaggcggaggaacaagaagagccagtaccgcggcgtcaggcagcggccgtggggcaagtgggcggcggagatccgcgacccgcgccgcgccgtgcgggtgtggctcgggaccttcgacaccgccgaggacgccgccagggcctacgaccgcgccgccgtcaagttccgcggcccgcgcgccaagctcaacttctccttccccgagcagcatctccgcgacgacagcggcaatgccgcggccaagtcagacgcgtgctctccgtcgccttcgccccgcagcgcggaggaggaggaaacaggggacctgctctgggacggcctggtggacttgatgaagctggacgagagcgacctctgcttactgctcccggtcgacaacactttggataaatttcacgcaccgggacagagacgatcgggatcaggggtacccctctgctactagtgttagactattagcgaggacagtaccagatagactggtgtcagtgcgcttgtacgccactcctaatcctgctcactgcttggttagcacacgtcctagcttcggttgtaattctgcatgcataaataggcacccgatc

SEQ ID NO: 8 - protein, 256 aa, Zea mays
  misc_feature: CeresClone ID no. 1494990
MEASRQYMIRFDGHFEEGPSSAAAEPPQPFASRAFSPEQEQSVMVAALLHVVSGYATPAPDLFFPAGKEACTACGVDGCLGCEFFGAEAGRAVAASDAPRAATAGGPQRRRRNKKSQYRGVRQRPWGKWAAEIRDPRRAVRVWLGTFDTAEDAARAYDRAAVKFRGPRAKLNFSFPEQHLRDDSGNAAAKSDACSPSPSPRSAEEEETGDLLWDGLVDLMKLDESDLCLLLPVDNTLDKFHAPGQRRSGSGVPLCY

SEQ ID NO: 9 - DNA, 1139 bp, Triticum aestivum
  misc_feature: Ceres Clone ID no. 634402
aaccatcaccaccgagctacctccagcctccagccatcactcagctcaaagacacaatcaggcaatcagcggcggcgagaacacacacagagtcacagatgaccttcagcgtctcgccggcgacgggggcgagccaggagtacatgatccggttcgacggccacttcgaggacccgagctccgcggccgcgagcgccgagccacccctgccgttcgccggcagggctttctcgccacagcaggagcagagcgccatggtcgccgcgctgctgcacgtcgtctccgggtacaccacgccggcgcctgacctcttcttcccggcgcgcaaggaggcgtgcacggcgtgcgggatggacgggtgcctcgggtgcgagttcttcggcgcggargccgggcgcgcggtcgcggcatcggacgcgccgagagcgccggcggccggcgggccgcagaggaggcggaggaacaagaagaaccagtaccgcggcgtcaggcagcggccgtggggcaagtgggcggcggagatccgcgacccgcgccgcgccgtgcgggtgtggctcgggaccttcgacacggccgaggacgccgccagggcctacgaccgcgccgccgtcgagttccgcggcccgcgcgccaagctcaacttctccttccccgagcagcagcagcagcagctaggcggcagcggcaatgccgcggccaagtcagacgcgtgctcgccctcgccttcgccccgcagcgcggacgaggacgaaacaggggacctgctctgggacggcttggtggacttgatgaagctggacgagagcgacctctgcttactgctcccggtcgacaacacggataaatttcacatagaggggaagagacgatcaggatcaggggtacccctctgctactagtgttagactattagcgagtaccagatagacaatcagcagtcctaatcctgctcactacgtggttagcacacgtcctagcttcggttgtaattctgcataaataggcacccgatcaatggaaaagttgtgttcaaccatactcatctgccatgttgtatgtagacaaatccaatttggggcttatttgttggaactataatggtttctattaatagaaaccaggggaaaccccttttgcac

SEQ ID NO: 10 - protein, 266 aa, Triticum aestivum
  misc_feature: CeresClone ID no. 634402
MTFSVSPATGASQEYMIRFDGHFEDPSSAAASAEPPLPFAGRAFSPQQEQSAMVAALLHVVSGYTTPAPDLFFPARKEACTACGMDGCLGCEFFGAEAGRAVAASDAPRAPAAGGPQRRRRNKKNQYRGVRQRPWGKWAAEIRDPRRAVRVWLGTFDTAEDAARAYDRAAVEFRGPRAKLNFSFPEQQQQQLGGSGNAAAKSDACSPSPSPRSADEDETGDLLWDGLVDLMKLDESDLCLLLPVDNTDKFHIEGKRRSGSGVPLCY

SEQ ID NO: 11 - protein, 275 aa, Oryza sativa
  misc_feature: GI ID no. 125603736
MGGNQEYMIRFDGHIDDASPSSATAEPPPPLPPPRPFAGRAISAEREHSVIVATLLHVISGYRTPPPEVFPAARAEVCGVCGMDQCLGCEFFAGESGVVSFDGAEKVAAAAAAAAAGAAAGQRRRRKKKNKYRGVRQRPWGKWAAEIRDPRRAVRKWLGTFDTAEEAARAYDRAAVEFRGPRAKLNFPFPEQLSAHDDSNGDASAAAKSDTLSPSPRSADADEQVEHTRWPQGGGGGGGGGGGETGDQLWEGLQDLMQLDEGGLSWFPQSSDSWN

SEQ ID NO: 12 - DNA, 849 bp, Oryza sativa
  misc_feature: Ceres Annot ID no. 6318302
atgaccaaccggatctccgccatgggaggcaaccaggagtacatgatccgattcgacggccacatcgacgatgcctcgccgagctccgccactgcagagccaccgccgccgctgccgccgccgcgtcccttcgccgggagggcgatctcggccgagagggagcactctgtgatcgtcgcgacgctgctccatgtcatctccggctacaggacgccgccgccggaggtgttcccggcggcgcgcgcggaggtgtgcggggtttgcgggatggaccagtgcctcgggtgcgagttcttcgccggggagtccggggtggtgtcgttcgatggcgcggagaaggtggcggcggcggccgccgcggcggcggctggcgccgcggcggggcagaggaggaggaggaagaagaagaacaagtaccgcggcgtgcggcagcggccatgggggaagtgggctgcggagatccgcgaccctcgccgagcggtgcgcaagtggctggggacgttcgacaccgccgaggaggccgccagggcgtacgaccgcgccgccgtcgagttccgcggcccgcgcgccaagctcaacttcccgttccccgagcagctctccgcgcacgacgacagcaatggcgacgccagcgccgccgccaagtccgacacattgtctccgtcgccgcgcagcgcagacgccgacgagcaagtagagcacacgcggtggccgcagggaggaggaggcggcggcggcggcggcggcggcgagacaggggaccagctctgggaaggcttgcaagacctgatgcagctggacgaaggcgggctcagctggttcccacagtcgtcagattcttggaattga

SEQ ID NO: 13 - protein, 282 aa, Oryza sativa
  misc_feature: Ceres Annot ID no. 6318302
MTNRISAMGGNQEYMIRFDGHIDDASPSSATAEPPPPLPPPRPFAGRAISAEREHSVIVATLLHVISGYRTPPPEVFPAARAEVCGVCGMDQCLGCEFFAGESGVVSFDGAEKVAAAAAAAAAGAAAGQRRRRKKKNKYRGVRQRPWGKWAAEIRDPRRAVRKWLGTFDTAEEAARAYDRAAVEFRGPRAKLNFPFPEQLSAHDDSNGDASAAAKSDTLSPSPRSADADEQVEHTRWPQGGGGGGGGGGGETGDQLWEGLQDLMQLDEGGLSWFPQSSDSWN

SEQ ID NO: 14 - DNA, 891 bp, Sorghum bicolor
  misc_feature: Ceres Annot ID no.  6014857
atgaccaaccgcgccttctccgccatggcagcgagccaccaatcatcctacatgatccgattcgacggcgcccactccgacgacccgtcgccgagctccgccggcgccgagccgccgggccccgcgcagccgcagccgcagccgcagccaccgttcgcggggcggaggatgatctcccccgagcaggagcaccaagtcattgtcgccgccctgctccacgtcgtctccgggtacaccacgccgccgccggaggtcttcccgcctccaccgccgccgacagcggcgtgctgccagctgtgcgggatggagcggtgcctcggctgcgagttcttcgccgccgccggggagggctgcttattaccagcgacgaccgcattggacggcgcggggaaggcagtggccgcggcgacaagcgcggcgccggggcagaggaggcggaggaagaagaagaacaagtaccgcggcgtgcggcagcggccgtgggggaagtgggcggcggagatccgcgacccgcgccgcgccgtgcgcaagtggctgggcacgttcgacaccgccgaggaggccgccagggcgtacgaccaggccgccatcgagttccgcggcccgcgcgccaagctcaacttcccgttccccgagcagctggcgacgggcacgggccacgacgaggccagcgcggccgccaccaccaagtcgtcggacaacacgctgtcgctgtcgccgtcgctctgcagcgacgagcgggagcgggagcgggggcagccggagtggctgccgagcgccgggctcggagggcaggaaacaggggagcagctctgggaaggcctgcaggacttgatgaagctggacgaaggcgagctctggttcccgccaacctcgagcgcttggaattga

SEQ ID NO: 15 - protein, 288 aa, Sorghum bicolor
  misc_feature: Ceres Annot ID no.  6014857
MAASHQSSYMIRFDGAHSDDPSPSSAGAEPPGPAQPQPQPQPPFAGRRMISPEQEHQVIVAALLHVVSGYTTPPPEVFPPPPPPTAACCQLCGMERCLGCEFFAAAGEGCLLPATTALDGAGKAVAAATSAAPGQRRRRKKKNKYRGVRQRPWGKWAAEIRDPRRAVRKWLGTFDTAEEAARAYDQAAIEFRGPRAKLNFPFPEQLATGTGHDEASAAATTKSSDNTLSLSPSLCSDERERERGQPEWLPSAGLGGQETGEQLWEGLQDLMKLDEGELWFPPTSSAWN

SEQ ID NO: 16 - DNA, 1078 bp, Panicum virgatum
  misc_feature: Ceres Clone ID no. 1824070
tcagttcaaagcagcagcgcctcgccatcagagacaagcacacgagctcagagaagaacaccacacgcatgaccaaccgcatcttctccgccatggcaggggaccaagcgtacatgatccgattcgacggccacttcgacgaccccacgccgagctccgccggcgcggagccgctggcgatgccgcagccgccgccgttcgcggggcgggtgatctcccccgagcaggagcaccaggtcattgtcgccgccctgctgcacgtcgtctccgggtacaccaccgcgccgccggagatcttcccgcccgccgccgcggcgtgccgggtgtgcgggatggagcggtgcctcggctgcgagtttttcggggcagagggcgccgcggcgatcgcattggacggcgcggcgagtaatgtggccggcgcgccgggcgcggcggcggcggcagggcagaggaggcggcggaagaagaagaacaagtaccgcggcgtgcgccagcggccgtgggggaagtgggcggcggagatccgcgacccgcaccgcgcggtgcgcaagtggctcgggacgttcgacaccgccgaggaggccgccaaggcctacgaccgcgccgccatcgagttccgcggcccgcgcgccaagctcaacttcccgttcccggagccgcccgcgggccacgacgaggccagcaacggcgacgcgagcgcggccgccaagtcctcggacaacacgctgtcgctgtcgccgtcgctctgcagcggggacgccgaggagcgggggcagccggcggagtggccgctgggcgggcaggaaacaggggagcagctctgggaaggcctccaggacctgatgaggctggacgaagccgagctctggttcccgccaacttcgaacgcttggaattgaaacgtgcgcgcgagtagatcctagccgttcaagtggttccaaaacgaacatcgtagcctttcattatgactttttttaccagctctgttgtaattatttgatcatagcagagttttgtaaattatgtgtagagtccaaccaactctatgaatcaggcaattttgggagcgtgcctttct

SEQ ID NO: 17 - protein, 268 aa, Panicum virgatum
  misc_feature: CeresClone ID no. 1824070
MAGDQAYMIRFDGHFDDPTPSSAGAEPLAMPQPPPFAGRVISPEQEHQVIVAALLHVVSGYTTAPPEIFPPAAAACRVCGMERCLGCEFFGAEGAAAIALDGAASNVAGAPGAAAAAGQRRRRKKKNKYRGVRQRPWGKWAAEIRDPHRAVRKWLGTFDTAEEAAKAYDRAAIEFRGPRAKLNFPFPEPPAGHDEASNGDASAAAKSSDNTLSLSPSLCSGDAEERGQPAEWPLGGQETGEQLWEGLQDLMRLDEAELWFPPTSNAWN

SEQ ID NO: 18 - DNA, 1090 bp, Panicum virgatum
  misc_feature: Ceres Clone ID no. 1805402
accagttcccgctcagttcaaagcagcagcgcctcgccatcatcagagacaaacacacgagctcagagaaggaaacaccacacgcatgaccaacggcatcttctccgccatggcaggggaccaagcgtacatgatccgattcgacggccacttcgacgacacctcgccgagctccgccggcgccgagccgccggaggtgcaggtgcaggtgcagcagccgccgccgttcgcggggcgggtgatctcccccgagcaggagcaccaggtcattgtcaccgccctgctgcacgtcgtctccgggtacacaaccgcgccgccggagatcttcccgcccgccgccgcggcgtgccgggtgtgcgggatggagcggtgcctcggctgcgagggcgccgcggcgatcgcattggacggcgcggagagcaatgcggccgcggcgccgggcgcggcagggcagaggaggcggaggaagaagaagaacaagtaccgcggcgtgcgccagcggccgtgggggaagtgggcggcggagatccgcgacccgcgccgcgcggtgcgcaagtggctcgggacgttcgacaccgccgaggaggccgccaaggcgtacgaccgcgccgccgtcgagttccgcggcccgcgcgccaagctcaacttcccgttccccgagcaggccgcggggcgcgacgaggccaccagcaacggcgacgcgagcgcggccgccaggtcctcggacaacacgctgtcgccgtcgctctgcagcggggacgccgaggagcgggggcagccggcggagtggccgcggggcggggggcaggaaacaggggagcagctctgggaaggcctccaggacctgatgaggctggacgaagccgagctctggttcccgccaacttccaacgcttggaattgaaacgtacgcgcgattagatcctagccgttcaagcggttccaaaatgaacatcctagcctttcgatgtgacttttttttttccagctctgttgtgcttatttgatcatagcagagttttgtaaattacctgtagagtccaaccaactgtattaatcaggcaattttgggagtgtgcctttctaccgc

SEQ ID NO: 19 - protein, 272 aa, Panicum virgatum
  misc_feature: CeresClone ID no. 1805402
MTNGIFSAMAGDQAYMIRFDGHFDDTSPSSAGAEPPEVQVQVQQPPPFAGRVISPEQEHQVIVTALLHVVSGYTTAPPEIFPPAAAACRVCGMERCLGCEGAAAIALDGAESNAAAAPGAAGQRRRRKKKNKYRGVRQRPWGKWAAEIRDPRRAVRKWLGTFDTAEEAAKAYDRAAVEFRGPRAKLNFPFPEQAAGRDEATSNGDASAAARSSDNTLSPSLCSGDAEERGQPAEWPRGGGQETGEQLWEGLQDLMRLDEAELWFPPTSNAWN

SEQ ID NO: 20 - DNA, 885 bp, Sorghum bicolor
  misc_feature: Ceres Annot ID no. 6041905
atgaccaagaagctcatctccgccatggccgggaagcaaggtttcaaggagcagcagttcaatgatcagaggagacagcaggcttcgattcaaggagacgacatcgccaagagcctggtggggttcggcggcggcggcggcaggctgatctcccatgagcaggaggacgccatcatcgtggcggcgctgcggcacgtggtgtccgggtacagcacgccgccgccggaggtcgtcacggtggcgggcggcgagccgtgcggggtctgcggcatcgacggatgcctcggctgcgacttcttcggggcggcgccggagctgacgcaacaggaagcagtgaacttcggcacagggcagatggtagcgacagctgcggcagcggcggccggaggggagcacgggcagaggacgcggcggcgtcggaagaagaacatgtaccgcggcgtgcggcagcggccgtgggggaagtgggcggcggagatccgcgacccgcggcgcgcggcgcgcgtgtggctgggcacgttcgacaccgcggaggaggccgccagggcctacgactgcgccgccatcgagttccgcggcgcgcgcgccaagctcaatttcccgggccacgaggcgttgctgccgttccagggccatggccatggcggcgacgcttgcgccaccgcggcggcgaacgccgagacgcagacgacaccgatgctgatgacgccgtcgccgtgcagtgcagacgccgcggcggcggcgccgggagactggcagctgggcggcggcgtggacggcggagagggagacgaggtgtgggaaggtctgctacaggacctgatgaagcaggacgaggcggacctctggttcttgccattttccggcgctgcatctagtttttga

SEQ ID NO: 21 - protein, 286 aa, Sorghum bicolor
  misc_feature: Ceres Annot ID no. 6041905
MAGKQGFKEQQFNDQRRQQASIQGDDIAKSLVGFGGGGGRLISHEQEDAIIVAALRHVVSGYSTPPPEVVTVAGGEPCGVCGIDGCLGCDFFGAAPELTQQEAVNFGTGQMVATAAAAAAGGEHGQRTRRRRKKNMYRGVRQRPWGKWAAEIRDPRRAARVWLGTFDTAEEAARAYDCAAIEFRGARAKLNFPGHEALLPFQGHGHGGDACATAAANAETQTTPMLMTPSPCSADAAAAAPGDWQLGGGVDGGEGDEVWEGLLQDLMKQDEADLWFLPFSGAASSF

SEQ ID NO: 22 - protein, 266 aa, Oryza sativa
  misc_feature: GI ID no. 115479555
MAAARQDSCKTKLDERGGSHQAPSSARWISSEQEHSIIVAALRYVVSGCTTPPPEIVTVACGEACALCGIDGCLGCDFFGAEAAGNEEAVMATDYAAAAAAAAVAGGSGGKRVRRRRKKNVYRGVRHRPWGKWAAEIRDPRRAVRKWLGTFDTAEEAARAYDRAALEFRGARAKLNFPCSEPLPMPSQRNGNGGDAVTAATTTAEQMTPTLSPCSADAEETTTPVDWQMGADEAGSNQLWDGLQDLMKLDEADTWFPPFSGAASSF

SEQ ID NO: 23 - DNA, 825 bp, Oryza sativa
  misc_feature: Ceres Annot ID no. 6325681
atgaccaagaaggtgataccggccatggcggcggcgaggcaggattcttgcaagaccaagcttgatgagcgtgggggtagtcatcaggctccgagctccgcgcggtggatctcgtccgagcaggagcacagcatcatcgtcgcggctctgcggtacgtggtgtccgggtgcaccacgccgccgccggagatcgtcacggtggcgtgcggggaggcgtgtgctctgtgcggcatcgacggctgtctcgggtgcgacttctttggggccgaggcggcggggaacgaggaggcggtaatggcgacggattatgctgctgctgctgctgcggccgcggtggcaggaggatcaggcgggaagagggttaggcggaggaggaagaagaacgtgtaccgcggcgtgcggcatcggccgtgggggaagtgggcagcggagatacgcgacccgcgccgcgcggtgcgcaagtggctcgggacgttcgacaccgccgaggaggccgccagggcgtacgaccgcgccgccctcgagttccgcggcgcgcgcgcgaagctcaacttcccgtgctccgagcctttgcccatgcccagccaaagaaacggcaatggcggcgatgctgtcacggcggcgacgacaacggcagagcagatgactccgactctgtcgccgtgcagcgcggatgccgaggagacgacgacgccggtggattggcagatgggcgcggacgaagccggcagcaaccagctctgggatggcttgcaggacctgatgaagctggatgaagcggacacctggttcccgccattttccggtgcagcgtctagtttttga

SEQ ID NO: 24 - protein, 274 aa, Oryza sativa
  misc_feature: Ceres Annot ID no. 6325681
MTKKVIPAMAAARQDSCKTKLDERGGSHQAPSSARWISSEQEHSIIVAALRYVVSGCTTPPPEIVTVACGEACALCGIDGCLGCDFFGAEAAGNEEAVMATDYAAAAAAAAVAGGSGGKRVRRRRKKNVYRGVRHRPWGKWAAEIRDPRRAVRKWLGTFDTAEEAARAYDRAALEFRGARAKLNFPCSEPLPMPSQRNGNGGDAVTAATTTAEQMTPTLSPCSADAEETTTPVDWQMGADEAGSNQLWDGLQDLMKLDEADTWFPPFSGAASSF

SEQ ID NO: 25 - protein, 259 aa, Raphanus raphanistrum
  misc_feature: GI ID no. 154093739
MHYPYTRPGFIGASDTQTRYSYQEQLSREQELSVIVAALQHVISGGSETTPYLGFSSDSTVIMPRSDSDTCQVCRIDGCLGCDYFFAPNRRIEKRQVEEEDGVTSNSSGREGSLTAAKKAEGGKIRKRKNKKNGYRGVRQRPWGKFAAEIRDPKRSVRVWLGTFETAEDAARAYDRAAVGFRGPRAKLNFPFMDYTSSTSHVENESTSVSARASASVSAGSSVEAEQWCGCECDMDEYLKMMMMMDFGSGDSSDSETHC

SEQ ID NO: 26 - protein, 262 aa, Brassica rapa
  misc_feature: GI ID no. 156950515
MHYPNNRPELSSGAPSPDRDLTRYPYHNQEQELSVIVSALQHVISGENETAPYQGFSSTVISAGMARSDSDACQVCRIDGCLGCNYFYAPNQRIENRHHQQVEAEEEGVSNSRRESHVAAAEGGGKVRKRKNKKNGYRGVRQRPWGKFAAEIRDPKRATRVWLGTFETAEDAARAYDRAAIGFRGPRAKLNFPFADYTSSADDVGTSASANASTSVSATESAEAEQWRGGDCDMDEYLKMMMMDFGNGDSSDSGNTIADMFQ

SEQ ID NO: 27 - protein, 261 aa, Nicotiana tabacum
  misc_feature: GI ID no. 129560507
MQRSTKRSKQEETVSINHLISKPKFTDEQEFSIMVSALTNVITGDTTQEFQYIISSSSPSTSMYSFDPPLFRVPKEPEPCQFCKIKGCLGCKYFGAPDPVAAAAAADNNNKAKIVAKKKKKNYRGVRQRPWGKWAAEIRDPRRAARVWLGTFNTAEDAARAYDKAAIQFRGPRAKLNFSFADYKSIQQHNTTTSISCSKQQQQEPIQLEQGIKTDVGIGKDEEFWDQLMKWDNEIQDCLNIMDFNGHSSDSAGGSIAHSFR

SEQ ID NO: 28 - protein, 283 aa, Nicotiana tabacum
  misc_feature: GI ID no. 129560505
MQRSNKRFREDGTSNTDQNNQQFPHFPRLTGEEEYSVMVSALKNVINGSIPMENTQQFYSFSPFQYCTATSTATTVTAYSSPSNSMSTIEQGNVVSPILGVPAEQEPCPFCRIKGCLGCDIFGTTSNAAAVVAADDNKKNSTTTTAVTKKKKKNYRGVRQRPWGKWAAEIRDPRKAARVWLGTFNTAEDAARAYDKAAIEFRGPRAKLNFSFADYTEIQEQQSASSSSPQQLPEPQLQQGNNTEFGNEIWDQLMGDNEIQDWLTMMNFNGDSSDSTGGNVHSV

SEQ ID NO: 29 - protein, 247 aa, Vitis vinifera
  misc_feature: GI ID no. 157341002
MQQRTPKRQKHASAPLSAGETASLQSPPQRLTPEQEGAIIVAALKTVISGGDAQDFRLFPSSMDCATTSTDVHGNAFLPISDPEPCQFCKIKGCLGCNFFQEDSKSVSVATTTKKKKKNYRGVRQRPWGKWAAEIRDPRRAARVWLGTFDTAEAAARAYDKAAIDFRGPRAKLNFPFPDNTLLTQNTVETEQPLQENQGNSEFLAQTGDINENGFWEMIGNDQWMTMVGFTGGDSSDSATTGNAHSF

SEQ ID NO: 30 - DNA, 801 bp, Populus balsamifera
  misc_feature: Ceres Annot ID no. 1460991
atgcaaagatctccaaagaggcccaaaatcaatgaagctccgtcagcgactctcttttcaccgccggcagctccaccgctaagattgacccaagagcaggagttggccgtgatggttgctgctctcaaaaacgtagtttctggcaccgcttcaatggatttctcaagggagatgaatagtattaatatgccaatcatcacttcacatccacaatttggaagtgcaagcaataacgggaatggtttttgcaactctatattgcctccatcttcggatcttgacacgtgtggtgtttgcaagatcaaagggtgcttaggatgcaactttttcccgccaaatcaagaagataaaaaggacgacaagaaagggaaacgaaagagagtaaagaagaattatagaggtgtaaggcaacggccatggggaaaatgggctgcagagataagagatccacggaaagcggcaagggtttggttagggacgtttaacactgcagaggaggcggcaagggcttatgataaggcagccattgattttagagggccaagagctaagcttaattttccatttcctgatagtggtattgctagttttgaagagagtaaagaaaagcaagaaaagcagcaggaaatcagtgagaagagaagtgaatttgaaacggaaacggggaaagacaatgagttcttggataatattgtagacgaagagttacaagaatggatggcgatgattatggattttggtaatggtggttcttccaattcttccggtaccgcaagtgctgctgctaccattggtttttaa

SEQ ID NO: 31 - protein, 266 aa, Populus balsamifera
  misc_feature: Ceres Annot ID no. 1460991
MQRSPKRPKINEAPSATLFSPPAAPPLRLTQEQELAVMVAALKNVVSGTASMDFSREMNSINMPIITSHPQFGSASNNGNGFCNSILPPSSDLDTCGVCKIKGCLGCNFFPPNQEDKKDDKKGKRKRVKKNYRGVRQRPWGKWAAEIRDPRKAARVWLGTFNTAEEAARAYDKAAIDFRGPRAKLNFPFPDSGIASFEESKEKQEKQQEISEKRSEFETETGKDNEFLDNIVDEELQEWMAMIMDFGNGGSSNSSGTASAAATIGF

SEQ ID NO: 32 - DNA, 475 bp, Panicum virgatum
  misc_feature (1)..(475): Chimeric switchgrass nucleotide sequence
ggagaaaggcatttcaaagatcagggcaaggaagagtgagctgctgtctgcagagatcaattacatggtcaaaagggagactgagctccagaatgaccacatgaaccggcacagcagacagcgaacatgatgggggagtcgtcgacgagtgagtaccagcaaggtttcattccttatgacccaataagaagcttcctgcagttcaacatcacgcagaaggccaccattgagaggtacaagaaggccaacagtgacacctccaactctggcacggttgcagaagtcaatgcccagcattaccagcaggagtctgccaagttgcgccagaccatcagtagcttgcaaaactcaaacaggaccttggtgggagatgcaatccaaaccatgagcctcagggatcttaagcagctggagggcaggctggagaaaggaatagccaagattagagccagaaagaacgagttgttatacgctg

SEQ ID NO: 33 - DNA, 2467 bp, Panicum virgatum
  misc_feature: Ceres Clone ID no. 1807588
cctctcgtcctcctcacccccaccccatggccatcaccaccacccactgctcctcctccgacctcgctacttatcgtgtctgcgctgctcctgctcctgctcctgctccggcgccatagagacgtgcatgcgcgcacgtagattggtcggagccgccgatcgagctcgagggtggggtaggtagtagtggagcaggaggtggtggcagactgactggctggtgagttgaagcttgagcgggaggaggaggtgcggactttgcctccggcgggccgcggatcggagatggtgctggatctcaatgtggcgtcgccggaagagtcgggtacgtcgagctcgtccgtcctcaactccggggacggcggcttccggttcggcctgctcgggagccccgtcgacgacgacgactgctccggcgagatggcgcagggcgcctcctcgggattcacgacgaggcagctcttcccgccccccgaccccgccggccgagccggagcgggcggcggcgccggtgccggtgccggtgtggcaaccacgccgcgccgaggatctcggcgccgcgcagaggccggtggcggccgcgaagaagacgcggcgcgggccgaggtcgcgtagctcgcagtacaggggcgtcaccttctacaggaggacgggccgctgggagtcgcacatctgggattgcgggaagcaagtctacctaggtggctttgacactgcgcacgcagctgcgagggcgtacgatcgagctgcgatcaagttccgcgggctcgaagctgacatcaacttcagtctgggcgactacgaggatgatctgaagcagatgaggaattggaccaaggaggagttcgtgcacatactccgccgacagagcacggggttcgcgagagggagttccaagtaccgtggcgtgacgctgcacaagtgtggccgctgggaggccaggatgggccagcttcttggcaagaagtacatctatcttgggctctttgacagcgaagttgaagctgcaagggcatatgacagggcagcccttcgcttcaatgggagggaagctgttactaacttcgagcctagctcctacaatgcaggagatgccctgcccgacaccgaaaatgaggcaattgttgatgctgatgccgttgatttggatctgcggatttcacaacctaatgtgcaagaccctagaatggataatactttagctggcctccagccaacatgcgactcccctgaatcatcgaatatgatgactactcagccaatgagctcatcgtccccgtggcctatgtatcaccaaagccaagcagtaccatctcacggtcagcgcttgtactcatcagcttgtgctggcttctttccgaaccatcaggaaaggaccatgatggagcgaaggcctgagctgggcgcccagcagccgttccccccctgggcatggcaaatgcagggctcccctcacatgccgttgcaccactctgcagcatcatcaggattctctaccgcccccggcggcgcgagcggcggcgtgccgttgccgtctcgccctccggcgacgcagttcccgaaccaccaccaattcttcttcccctaggccgcttgacacttgacagcctgctgctccggctcaattccttgggcctctgggacggcaggagctgatcttgtgcagcttgcttcccggtaacggttgtttattattgggaggaaagcgagagccagaagacgcagtatggctcgtctccctccctggtacggtatgttatggtatggtgcccttgctaaaatatgggctattgctacaatgccttggatttgtcatggttgagatttttctacccatgcatcagtgtatttcttcttctaccggatcatggatgtacacaagctgcttcaggagtttgactccttttgttttaaatttgcatgagaagtttcaagtttgcctttcaggatgagaggaaatggcaaatctgccagtactgttgcgttttacccgcagcagccgcggcgctagcgcagcccccctggcccggcactgtagccggcccccgggtccgggtggtggcgccggctgacatgcgggccgcctctggggaaaggggactgggcctcgtacgttccctcggctgtcagcggctgtccgttggcgttgcgattggcacccaccactttgggaggggtgggggagcagctcgccagctcctgatggccgcacagtgaagtgaaagctagtggtaatacctagccttgtggagctagccatggggtgtttagttggtgaaaaaaatttgagtttggctactatagtatattttattgttatttgataattaatatttaattatgaattaattagcgtcattaggtatatatcgtaggaattaattaaactatataattagttatttttttcaacagtatttaatgctttatgcatttgatgtaagaagtattgcgcaaattattttgg

SEQ ID NO: 34 - DNA, 1275 bp, Panicum virgatum
  misc_feature: Ceres Clone ID no. 1821199
agaacgggagcggcgaggtcgagctcctactgaggctgcgtgaggccgagagcccgcccgagatcaatttcggcaataattgcagctcgagcaggcaaatcatatacagcttgggtgagaggtgagaagagagaagcgagggtgagatcgaccggccgatcgggagggcaggccatggggcgcgggaaggtgctgctgcagcggatcgagaacaagatcagccggcaggtgacgttcgccaagcgccggaacggcctgctcaagaaggcctacgagctctccatcctctgcgacgccgaggtcgcgctcgtcctcttctcccacgctggccgcctctaccagttctcatcctcctccaatctgcttaaaactttggagaggtaccagaggtacatctatgcttctgctgatgctgcagcgccgtctagtgatgagatgcagaataactatcaagaatatatgaagttgaagacaagagttgaggctttacaacgctcgcaaaggaatcttctgggtgaagacctggctccacttaccaccagtgaacttgaccagcttgagagtcaagtagacaagaccttgaagcaaatcagatcaagaaagactcaagtgctacttgatgaactctgcgacttgaagagaaaggaacatatgctccaagatgccaacatggtcctgaaaagaaagctggacgaggtcgaggcagaggcgcctcctcgcccacagccacagctgccgtggcagggcggcagcggtgatggcaccatggtgtcggacggccctccacagccagagcacttctttcaggccctggagagcaacccatctctgcagccaacgttccataccatggacatgaaccagcagccggtgccagcgccgggcggctcctactctcctgcgtggtcggcgtggatggcatagccagtgctttattgttgcattggccggagaagtgattgtgtcctccagagattcctacgagttccgtttctgcttttgctccccaatttgctcggcttccagcgcttgccgaaatgtaagtgacaacttaaaatagccctgtatagattgctgtccttatctaagttggtctctgctcagctgcttcattaagaggtgtgacgatgcatgataagagaaaaatggttgaacttcctatgttttggattctacattgcttgacaataagcactgtcatgttggactgctagactgcccatcgtttgacaatatttgtctttttttttcaaaatataatgcgtttggactgcc

SEQ ID NO: 35 - DNA, 1863 bp, Panicum virgatum
  misc_feature: Ceres Clone ID no. 2009001
accattcgtccccctcacgtcccgtcgcgccgcccccttcgtcctggggcgccctcctcctccatggccaccgcgccccgacgccgctgctaggttgctggtagaggaggaggaggagtccggggcagcaggcagcgcgcgagctcgatcgcgtgccggggccgagctggagccgatggtgctggatctcaatgccgccgactcgccgacgcccgggtcggcctcggccacctcgagctccagcggcgccgggggcttcttccggttcgacctgctcggcgggagccccgacgaggagggctgctgctcgccctcgccgcccgtcgtcacgcgccagctcttcccgtcgccgcacccggacgccgcctccgtcgccgccgcctcgccgccgccggacgggccgccgcccccggaggcgtcggggccctgggcgcgccgcgcggcggatttcgcgccttcgtcgcccgcggccgggaagaagagccgccgggggcccaggtcccggagctcgcagtacaggggcgtcaccttctacaggaggaccggccgctgggagtcgcacatctgggactgcgggaagcaggtctacctgggtggattcgacaccgcgcacgccgcagcaagggcctatgaccgggctgcgatcaagttcaggggcctcgacgcagacatcaacttccatctgaaggactatgaggctgacttgaagcagatgaagaattggaccaaggaggagtttgttcacatactccggcgtcaaagcactgggtttgcgagggggagctcaaagtaccggggtgtgactcaacacaagtgtggtcgatgggaagctcggatgggtcagcttcttgggaagaagtacatctacctcgggttgtttgacagtgaaatcgaagcagcaagagcatatgacagggcggccattcgtttcaatggtcccgacgctgttactaattttgattctagttcctatgatggagatgttccacttccacctgaaatcgagaaagatgtggttgatgaggacatccttgatttaaatttgaggatttcacaacctaatgtgcactttccgaaaagtgattgtatcctgactgggtttggagtaaactgtaattctcctgaagcttcaagttcaattgtttctcagccaataagccctcagtggcttgcacatccccacagcacattggttccaccccagcagccacatctgtatgcatctccttctccaggcttctttgtgaacctcagggaggaggcaccagcagccacggagaagcgcccggagccggggccgggtccccaggcgtcgttccctccttgggcgtggcaaatgcagggctcccctgcgccgttgctccccgccgctactgcagcatcatcaggattctctaccgccgccggcgcgccggcgccgtccggcccccgcccgttcgccggccgccgcggccaccaccaccagctccgcttccccccgaccgcctgacgggcgagctcctcctcctgccctgccctgccccgcccctatccatcccgagacgggcgcctggcctgaccggtgcccgtccgtcgctggtctggcctggtcggtgacaggggggccggccattggttgggggaccaggggacggactgaggcagagtccttcctctgtctccccagctggtcactaccgtcgaagctccggcgctcatggctccctcaagtccggatcttcgttttgggaagcggattcatggaccgtggccccgtttgatttggactaggaaagtttggagtagaaattttatcaactttgaccattaattacggtgtaaaataaagtcggtttataaaactaatttcagaactcttgcgtt

SEQ ID NO: 36 - DNA, 740 bp, Panicum virgatum
  misc_feature: Ceres Clone ID no. 1822499
aaagatacttgagagtgagagggagagagagaaggaaagatgggtgaccaacacatctagagagagtgagcgagggcgggggagaggggcggtgaaaactctctccaaaagcaccaaaaagacctcgccccaagggtacgatcgcataacaagcatctctttggttaaaacaagccagattccaggtgagcgagagagaacttgctttatagccagagggagaaagagagaggagaaggagggcgagagtaaggaagatggggcgcgggaaggtggagctgaagcggatcgagaacaaaatcagccggcaggtgacgtttgccaagcgcaggaacggcctgctcaagaaggcgtacgagctctcgctgctgtgcgacgctgaggtcgcgctcatcatcttctccggccgcggccgcctcttcaagttctccagctcgtcatgcatgtacaaaacacttgagagataccgcacctccaattacagctcacaggaaataaaaactccattggatggtgaaatcaactaccaggattatttgaagttgaagacatgagttgaatttcttcaaactacacaaagaaatattcttggtgaggatctgggtccacttatgatgaaggagcttgagcagcttgagaaccaaatagagatatccctgacacatatcacgacaagaaagaatcaaatgttacttgatctgctctttgatctgaaaagtaacgagcaagaattacaggac

SEQ ID NO: 37 - DNA, 1545 bp, Panicum virgatum
  misc_feature: Ceres Clone ID no. 1815457
gctttaaatccggcgcctcgccgcctccctcctcctccccgccccggccggtcaccccgcctgcctctctctctctccgccgccgcggccgtgacgtcatgcgctcgccggcgctgctgccgcctaccatccacaaccagccagccgtaggcttgtatctagccaatcagccctcgtaggtggcctgttttatagctgctgccgtcgttgtcgtcgcggctgaggcgacgtgaggattggttagagagggaaccaggatttggttgagaggaggcggcggcggcggcggcggcggcgatggggcgcgggaaggtgcagctgaagcggatcgagaacaagatcaaccgccaggtgaccttctccaagcgccgcgcggggctgctcaagaaggcgcacgagatctccgtgctctgcgacgccgaggtcgcgctcatcatcttctccacgaaggggaagctctacgagtacgccaccgacacaagtatggacaaaattcttgaacggtatgaacgctactcctatgcagaaaaggttctcatttcagcagaatctgaaactcagggcaactggtgccacgaatatagaatgctaaaggcgaaggttgagacaatacagaaatgtcaaaagcacctcatgggagaggatcttgaaactctgaatctcaaggagcttcagcaactagagcagcagctagagagttcactgaaacatatcagatccagaaagagccagcttatgatggagtcaatttcagagcttcaacggaaggagaagtccctgcaggaggagaacaagattctgcagaaggagctcgcagagaagcagaaagcccagcgacagcaagcgcaatgggaccagactcaacaacaaaccagctcgtcttcctcgtccttcatgatgagagaagctcccccagcaacaaatatcagctaccccgtggcagcaggcgggagggtggaggggccagcagcgcagccgcaggctcgcattgggctgccaccgtggatgcttagccacatcagcagctgaactgaaggctttcctctcgcccgtctcggtgtgcaagcccaaaatccagcaacgcaacggtagtatgctcacccggctgcgccaatgctccacttgatgcatcattatcgccgatcttgtcgtgatcgctaccagcagcagcagcagtaagcaggggtttatcataatttgagcagcatataagttccaatcttcgctgtgtatattttgcttttgttcatcaccatttccgctgaggggaccgtacgatgaataatttcccccatgtaatatataatatgcgcagcatgaatgtcaatcgagcggtttgtcaattgagtcctgtacaagttgcatcattgcttgttgtattcacaagacacctgtgcctccgcactcaatcctatctgtaggctttgattcttttatgtatcttctgccattctataatggccctgtttagatacgcttataatccgcttagggcatgagcaatgtattttacctatatcgatacacatgggtcatgtgtcagcc

SEQ ID NO: 38 - DNA, 2294 bp, Panicum virgatum
  misc_feature: Ceres Clone ID no. 1789568
gcacccgtctcgcctctgatcccatccttcttcctcgactccgtcggccaccgccaccgctcgccggcgccggccgtccgagctcggaaaggtccctcctttcctgccaagatcccacctttctttctcgagccagctgaccccgcctgctccgtactagctagctagttgattggtggggcgttcttgattcgtgaagcgaccgtacagtaggatcgtgctcgcgccgcctgagggttgcgtgcaggatggtcgtcaaagattgggagtgaggaggcagctcaacgcagtcgtggaggctaaatgtaccgcaagaacgactcggcactctcctgcttctacctcttcctcctctggttcttcttcttgaaatagaccagcccggcccagcgagagcaactcgactgcgattgagatcgatcgctgtctctcgttgtttggtccgtgtgaggctgaggtgattctttcctggctgtctgctccagcaagaatcgcaaggaagggaggagatggaactggatctgaacgtggccgaggtggcgccggagaagacggcggcgatggccacgagcgactccggctcatcggagtcgtcggtgctgaacgcggaggcgtccggcgggggagccgcgccggcggaggagggctccagctcgatgcccccgccgcccgccgtgctcgagttcagcatcctcaggagcgagagcgacgcggccggcgccgacgacgacgacgacgccacgccgtcgccaccgcaccaccaccaccagcagcagcagccgcagctcatcacccgggagctctttccggccgccgcgggcccgccgcgcccgccgccacagcattgggccgaccttggcttcttccgcgccgagccgccgctcccgcagccggacatcaggatcctgccgcacccacacgccacgccgccggcgccggcgcccgtgcagccacaggcggccaagaagagccgccgcggcccgcgttcccgcagctcgcagtaccgcggcgtcaccttctaccgccgcaccggccgctgggagtcccatatctgggattgcggcaagcaagtgtacttaggtggatttgacactgcccatgctgctgcgagggcgtacgatcgagcggccatcaagttccgcggcgtcgacgcggacataaacttcaatctcagtgactacgaagacgacatgacgcagatgaagagcctgtccaaggaggagttcgtgcacgtcctgcgaaggcagagcacgggattctcgcggggcagctccaagtacagaggcgtcaccctgcacaagtgcggccgctgggaggcgcgcatggggcagttcctcggcaagaagtacatatatcttgggctattcgacagcgaagtagaggctgcaagggcttatgacaaggccgcgataaaatgcaatggtagagaggccgtgacgaacttcgagccaagcacatatgacggggagctgctatccgaagttggaactgaagcgggtgcggatgttgatctgaacttgagcatatctcaaccggcatcccagagtccgaaaagagataagaactccctcggtctgcaactccaccatggatcctttgagggctccgagttgaaaaggacaaaggttgatactccccatgaactggccggtcgccctcatcggttccctgttatgactgagcatccaccgatctggcctgctcaatctcaccccttctttacaaataatgagagtgcatcaagagatcttaacaggaggccagcagagggggggacagggggtgttcccagctgggcatggaaggtgacagcccctcctcccaccctcccattgccgctcttgtcgtcgtcgtcgccgtccgctgcagcatcatcaggattctccaataccgccacgacagctgcccttgccaccccatcagcctccctccggttcgacccgccgtcgtcgtcgagccatcgccgctgaaaatcaagaagccacgctgtaaatttgccgggaagctggcatttttcccccctctgggcgttgcaactttttcggttttgcgcctgggtggtttcttgtagtggattggattcgtaactgcattttcataccgctcaagtgaaatggttctctctttagacactctgcatgctgctctgggagttgctgctgctggagattgactaacttcaacctctgagattgatctactatacattgtgtagagaatcattgctgaactattaacatacagaagtataggtatcataagcccatgtctgcctctacttgtggacagagttc

SEQ ID NO: 39 - DNA, 1264 bp, Panicum virgatum
  misc_feature: Ceres Clone ID no. 100174842
tcgcggatccgaacactgcgtttgctggctttgatgaaactcacatcctcttgcttccctctctggatctcttcctcatgagagatgcaaagaggcactgccataccatccatgctcgacatgatggctgatctgagctgcgggtcgtccaaggtgaaagagcagccggcgccgaccggctccgacgacaagccggggaggggcaagatcgagatcaagcgcatcgagaacacgaccaaccggcaggtcaccttctgcaagcgccgcaacggcctccttaagaaggcgtacgagctctccgtgctctgcgatgccgaggtcgcgctcatcgtcttctccagccgcggccgcctctacgagtacgccaacaacagtgtgaaggccaccattgagaggtacaagaaggccaacagtgacacctccaactctggcacggttgcagaagtcaatgcccagcattaccagcaggagtctgccaagttgcgccagaccatcagtagcttgcaaaactcaaacaggaccttggtgggagatgcaatccaaaccatgagcctcagggatcttaagcagctggagggcaggctggagaaaggaatagccaagattagagccagaaagaacgagttgttatacgctgaagttgagtacatgcagaaaagggagatggatctacagagtgacaacatgtacttgaggagcaaggtcgccgagaacaatgaaaggggacagccgcccatgaacatgatgggagcgccgtcgacaagcgaatatgatcacatggccccctacgactcgagaaacttccttcaagtgaacattatgcagcagcctcagcattactcccatcagctccaaccaacaacccttcagctcggatgaagaaaaattatccgaaggcgccggcgtcgacgcacgcaagcaactgttatcatgtgtccaatcgagatcacgtgaccctaaacgtttgtggaactagttaataatcatattgtaactagtagtacgagtgtgtgataactgtatgtaatttgtatccatacggcgaggttacgccttcagcgtctgcagcagcagagctcgtcgtcgatcaaccgcaaaagactatgcatctgtgcgagttaattaattaaaatgtcgtgtagcgcaatgtaatctgttcgtgttgtgtaataataaatctgaatccactaaatatttgctgcttttaattttctgcgcaaaaaaaaaaaaacctatagtgagtcgtattaattctgtgctcgca

SEQ ID NO: 40 - DNA, 2002 bp, Sorghum bicolor
  misc_feature: Ceres Promoter PD3579
aaactcttcgtcagtgctgatgacagaagcagctgcccttactctagcaaccacggtgctagaagctatgtacatgattgattccactattttaacagataatcaatagttagtactctttctaaacgggtcttagtttgatcatcatcctgatggagaattaaatcctacattcaaattaccagctccaagattcatggtacaactatagcgattcgcaagattaccagaatcatatggctgatcaactagctagataggctctgagtgaattagtttgcaatcaaatctctcttaatagtgcttgttgtcattctgctcatgagcaaaagtgtcctttactttcgacactctcaaatataactattaactctataatggtcctaaccgtaacacgctgttaatcatataggccttgttcagttggcaaaaattttgggttttaacactgtagcatttttgtttttatttgataaacattgtcagatgaactgtgtaattagtttttatttttatgtatatttaatgcaccatacatctgccgtaaaatttgatgggatggaaaatcttgaaaatttttgaaactaaacaaggccatagtttcattgtaaaaaaaaaaacagctaagcaagatggccgagagagccgttgacgcagagcattgaacggcatctctctcggctgctctcgaatgcgctgcctgccggcatcccggaaattgcgtggcggagcggagccgaggcgggctggtctcacacggcacgaaaccgtcccggcacacggcaccacgatttttccttcccctccccctgcccttctttttcctcataaatagccaccccctcctcgcctctttccccccaactcgtcttcgtccctcgtgttgttcggcgtccacggacacagcccgatcccaatccctcttctccgagcctcgtcgatcgcccccttccctcgcttcaaggtacggcgatcgtcctcccgctttcgcttctcccctcccctcctctcgattatgggttattggggctgcgagtcatctttctggcgatttattatggtctcgatctggtggtaactgtggcgatttattatgggagccctcgatctagaagtcgagtactctctctggtaactgtagcgatttgttatgggggctctcgatctagaagccgagtactctctggtaactgtgggacccttgtagggttgggttgttatgattatttgggcttgtgattaggttgtatctgatgcagaatgatgtattgatcgtcctattagattagatggaaacaagtagggtgactctgatttatttatccttgatctcgtttgatgtccctagctaggcctgtgcgtctggttcgtcatactagttttgttgtttttggtgctggttctgatgcccgtccagatcaagtcatatgaaccagctgctgtcttattaaatttggatctgcctgttttaacatatatgttcatatagaattgatatgagctagtatgaactagctgcttgtcttattaaatttggatctgcatgtgttatatgatggatgaaatatgtgcttaagatatatgctgcggttttctgccgaggctgtagcttttgtctgattaaagtgcatcatgcttattcgttgaactctgtggctgtcttaataagaattcatgtttgcctgatgttggagaaaacatacataagaattcatgtttgcctgatgttcgagaaaatatgcatcgacctacttagctattacttgatgcgcatgctttgtcctgttttgtttgatatgcatgcttagaaagattaaaatatatgtggctgctgtttgattcgataattctttagcatctacctgatgagcatgcatgctcttgttattcactgctactgttccttgattctgtgccacctacatgttacatgtttatggttgcttctttttctacttggtgtactactatatgcttacccttttgtttggtttctctgcag

SEQ ID NO: 41 - DNA, 1500 bp, Sorghum bicolor
  misc_feature: Ceres Promoter PD3800
catggaaccaaaggaaacacgtcaaaataatagcaggaaatatatgggcatcagaaagagtgcctgctgcctgtgactactagcttctacacttttcacaatgttgtctgtatctaaccactccatttctttcaaagctttgctggtttcttacacgcatgcaaggaggctattttcctttttcttttctagttcgttcggacgtctcatgtattgcgcaagcaagcacatgcactcatgaaaagctagcaaagacacatgatatggtggcttataaaaaaaagcacatgcattatattgttgtgtagcattttgacatgtatcataattgctactgtggtagtatcttgggtatagatcaccacacaacatttaatttaaaaggcccacggtcgattactagatacatattccttctgtgcaatacaagggattcaaatttgtcctaagtcaaattatcatgagtttgatctaatttaaagaaaagaacatataatatcaaactagtatcattagacacattgttacatacctctctttgctgatgtgatagatattaatactcttctttgtaaattctgtcgtactcagaatatatagtttaacttactttacatttcgggacagagggagtatatatatgttctgttcattctttgcctcgctccctcctttactcatcagtggcaggcaccttttcataatcttatataagtttgcgacacttggtaccgagcacacagcaccatcaccatcactacgtgcaagcaaaggcaacataacttatgtaggacccaattaaagacttaattaatgtagtatcatatttttcatcctactgtacctttttttagggggtgcggggggcttcttgatcactggcctatactgtactgattagtgatgtgtgtttcaccagattgtggctgctggtagtagtgacttggttgctcttgcatgtaacgacatttattgccataatgaatgaagtgctgtatgaaactactcaatgagggcagaggagaacattctaaaaattatttcctagctggaacacgcatttaatttagcacaacattccttccattggtcctaaggttattagggcacacaaatccaaacactacacttggagacttggagagaataatagaacagagagatgcatacaaatcatgcaagctcccagtagagtcctgtggactccttaacatttgctcctggaattgaatatggttaaacaaatgcaggtgcaccatgcatgtcacccctgcctgccatctcatctcatccacagtgcctgcccctgcatgccctccttcctttgctttccctcccaaaggacacctccaagctccatttaaataccacccctccctccctcacttgtgaccactactgcactacactactccaaaacgacctcaagccagcactcaacctaggtagctcacagccacagcagctaaagcctattagctcactcgtgctcatcttgcc

SEQ ID NO: 42 - DNA, 980 bp, Sorghum bicolor
  misc_feature: Ceres Promoter Annt 8643934
aaacgatgcaagcctcacgttgtgcagcacaacaccatcgacgactgcccttttatttaatttcgtgaccaaacggcccaaatgccgactgcatttttttctactctctactctcatcttcaataaactacattatgtatgtgtccatattaatttattagtttgagcatatttatatacttaatttaagatacttcaaatcttaatatgcgaaaaaattcagtacatctcatactttttaatataattactaaaatatcacttcaatagtatataaaataaatatatactatatttatacctacatatttaacatacgtactagtatgaccatacatatactctatcaacacatactttatcgggctatatacgtcataaatatatatatatatatatatatatatacacacgcgcgcgcgacgttgaaaaaaaactaggcctgcatgtgtttttttttggttgttcgaccgtacgaggagctcgctctcacgtagcccgatggaacccccatttattctcctttttcaacgcattttttcttgtacatatattagttaattaattaaggtcgagtaataagtagtaccactgggggaagagaaagagagagagaagcacaggcgctgcccgcagcagcgcgtcagcgcccgggacgacgagagagagagaggctgacaaggtgggcccgtgcgggccttgaccaatcggagttcgacagcagcctgcccccaaaaccacactcgctctcctcccctcgcgccgcggccgtcgcctcccctccctccaccgatcgatccctctcctcctcctcacatcccaccccccaccttctctttaaagctacctagctacctacctacctgccgcctcgccggcggccgctagctgccagtcgccaccgccgccgcatcgatcgatcggaacggaaggagctagctagcgcagcaagcgcccatcagcaagcagatcggagcaag

SEQ ID NO: 43 - DNA, 1000 bp, Sorghum bicolor
  misc_feature: Ceres Promoter Annt 8632648
ccggaccgcacaaacagaccgagccaggcgacctgttgctcccacttccccgttttgcccaaacattttcgtctcgttccgtcccgtagaccggcgccccccatccccccacgccgcagctttcttcacgtgcacggtgcacggcacgctgcgcttaaaaaggaaacaaaaacaaccagccgcaagctccaaaagatctgaaatctccaatgcttgggcacccggcaccactgggcaaatctgaaataatactaaaatccagtccacaaacaatctcgataccaaaatcgacaacaaagaatctagtgagatttcttaaatatatatatatacacactaatcaggactagtataggagtagtgcatattttttatatatagaaaaacaaaaacagataatagctgccaacaacttgtcgtgccaggctacgctgggagagagaagccggtttcgaccgtacgaggaaggaccttggccctggcggcgggcccatgcgatgagagatgcggtggggccctccgggcccgggcatggggcatcggccaatggctgttcgacagcggcggctcggaaaccatcccggtttcgcgataccccttcctcccctccgatccgtcgcggcagcgcgcatcaccgctttaaatccgccccctcccggcgcctccctcctcccggccggtccccctcaccttctctccctctcctctctctccagcctccaccgccgcagctagctagctgtgacgtcatgcactcgccggcgccatagcgcgccagctcctactatctacaactgtaggcttagttatcctgtcaatcaagcctctcgtaaggaacaaggaaggtagctagatagttttatagctgctgtcgtcgtcgtcggcggcggcggaagcgacgtacctgttcttagaggataggataggttagcagagagggtcagctagcgaggattttggttgagatcaggagggggaggaggaggaggaggcggcggcg

SEQ ID NO: 44 - DNA, 1500 bp, Sorghum bicolor
  misc_feature: Ceres Promoter Annt 8657974
ccaactctgatgtgtccacaatgccaccagcaacctgctatgtatcttgaaactgactgttcgttgataacatggattagagcagtcagtggagtggtagcttcacaacgacacattattgatcgatgaggatgtgcgtccactgtacatacatccatgcatattagtagtggcccaggtgagtgatggagttgggagggggcggtgggcaaggaatatgcatccgatccaccacattatgtaagggccccggttgggtttgggtcccattaattcatcccatgaggctatctccaacagggagacccatttgggacccaaacctaaaatgggtctccaacacaatacctatagcctccaacagagtacccatacagaagacctattttgggtatcaggagaggcataacccaaatttgggtatcctctctcctcgagacccatttgcagagagtgttgtcttttaggtcttgttgttggagaagactaaaaataggtatggaaccttttacctgtagcgctatccaaaggacaaatgggtcttgtattttgggtgacgattgttggagatagtctgagagcaacagaatgggagctgaaaataattgggatgctgtagctggatagtacttagcccatcatcccctgcatcatatatacacatatatgtgccaaacagaaatgggacagtgtttgtgcggtgtcacatgcggatagggcacacaatgttcctcatctatacatgcctgccttgatcacaagatattgaagaaaaaaagtgaagatgaaacaaaaagaatatgggataacagacactacagaattaacattttattagttcactagctcacaaaaaaagtaaatcattttatatgtataagtttgaattagaagatcaattttttattaatcattgtaaaaaactacagactttattataccggctgcctcatactatttaatcagtgagagtctgtaaaagaattgaaccgataaaaaaaggttcagaagcaaaactattgcaaattagttctaccaagccaatgagaattattgcaattcaggggggctagctaaagatgcactagcattgttgttacagttggggcttgctatatatgaacttctatcgttctctttcactctgcttggtgaattaaagtgcaaaaccttaaagagttaagcaagaaatagccagccttgctgcaccatatgcaaaacggtttttgtggtaattgtgtttgtgtgcctgacatatgcatgctgactgcagaggtagatggagatctgacaccaagcaagcacacacacacatacaagagggatcaatcaaaaaagtggtaaatcaaaaaggcttgtgcaagtcatcagaagcccagggggaacaaacaaaacaatcagcacaggccataggaattggccacagcccacagctgcacctgcatacagctgctgctaactgcatcacctcacataccttcccctctcttccttcctcag

SEQ ID NO: 45 - DNA, 1000 bp, Sorghum bicolor
  misc_feature: Ceres Promoter Annt 8681303
ctatctaaatttctcatctctcatctcatatcatgtaatggcagcatgcatggatctcatgatctcaatgatgtgtgttgtcgtcgtcatcgccgtcccatacatgcctacagaaatctggcacaaaggcgggcggctcacgtactagcatatacgctacaggcagcacacatgcaagtcgtactagcatgcaaccaagggaaaattggcaattggggttatggaatcaaacagggatctatatttttgggcagcctgcctccaccggtcggatcggagaagggttggatcatatcggaggcgcgcggcgtggcccaaagcgaaagcaacggcgcagggctgcagggttgagagcctgctgggacccaggaggtgtgtgaggtgcatggcgacctgcatggtttgggttaggcctaggaggttctctctccaatgggtagctcgcaccctttggccgccgttctcgtgtatgccatatgccatcatgctatgcttcgcgccgtgtatatttgtggcgtgggagccgccgcatcggagcgcccccgtttcggcacgggtctcccagttagggtaaaccaggggcagtgggtaaaactgcccagctccactccaaatttaccctccggcctctctcctttaatttaaagctagctctgagagatagatgggagtgcagagtgggggagatagatggatggatggagacgcgtgaaaaagatgcgatgcgagagaagagaccggcagcgtgctacagggcagccacacagtgacgctgccctttttcccggttctctccactgatattccgctcctgtcctgccccggcgacccattatatcgtcgcgcacaatgcaaaacttagcacttgtgggcggccttgttttgtggggagtcgtcgcctagctagcagcggataagcaggcagcagcagaagagcgagcagagagagcggctgagtgagagagagaacagcgagctctcggaggagaaggagatcgtc

SEQ ID NO: 46 - DNA, 1000 bp, Sorghum bicolor
  misc_feature: Ceres Promoter Annt 8732691
ctgatcgtgaatcttggtataggcacttaagtggaggtatgttgcaatgtctacacaaaatcaccgtcgcttgcaataacaaatacctatgatttaactatttctatatattgttgatcgtgaatcttgctacaggcacttaagtggatttctcatcctaagtatatgtctacatggaaaagaccaaaaaccacacacatttatacagcgagtataacaattttgtctatgtttttgtgaaagttttttcacaaaatgtagtattacccatgcgacacacactaatatttacatatatatatacatactcgaacgtgtgataggatggtacagagatatggtagaacttatttgtggatttacagtgcacgaaagaaatggatatcggagatttcttcctgccgatattaaatattatcttagtcgtatcacggaaatgtctataaagtagaatttatgagcctacaacgtcgtgctctctgtggctgtgttttatttcacaaactttgcttaactttgctttttaagataaatatcactttaaaattggtatttaagttttattataatattattattttattatgcgatccgtatattttaaataaaaatattagttattccctagcaacgtataggcacgctacatagtaaaagaaaaaagaaaatgattaaaaaaacataggccccaccgagcagcacaggctgcactacgtgcgaacgtaatcaacacagccacgctagccgtttccatttccggccctgcaatcctgcaccggcgcgaaagccccctcacggcctcacctccatcctccactccgtcgatggccgcctctcctttactcctcagtgctcacgccgtccacagtccactagtggcatcggctcccacataaaagatccacaccacttgagcagaaggctggtagtggaagggtaggctcttgcttgagctgagctcttgctgcctgtggatctctttgggagtggtgaacggagg

SEQ ID NO: 47 - DNA, 1000 bp, Sorghum bicolor
  misc_feature: Ceres Promoter Annt 8031970
catttgtaatgcatgtaagctgtggtccagaccttgtttggaacaaagttaaataaaaatacatgcatgaaacatgtacaatctaatttcgtttaattagattccattataaaatatacccactacattacttgacaacaacaattattcatatatatttcttctacaaacttaagaggcaatataaatgatgacccaataatatatggtgttgtgtagagtggacggggccagtgacgtgacgatgtaggtataaacaaataagagagctagctagctagcaggctactacggtgcatgctttgctttcagtccagtgcaaattaaaggggtgcatgcatgcatcagtgtggaggccaacaagatcgagaagaagcagtgcccaagaagaggatccggaagccaaaaccgtgctaaccgttgtgccaaaagccgccacggcagaccgaccgaccgaccacgaccagaccgatcgtcgacggatgcatggcacggcacggtggtggattgcaacgcacgcgcccagagcgagccggccgcgtcagatcacggggcagggggctgggcgcggccggcagtcgcaggcagcgacggccggggcgggcgggcagtgcacggcaccacacggcacggtgccccacgcctttcacggatccggtagctgtctccgtccacgccgcgcaccgcacctcgtcctctccaccccgaattgcacacgcacacaccttgtccttccatcccttgccgcaccaccgcccaccccctcctgcttattaccaccaccggctctctcttgtgctgtgctcagctcatctgcctctccatttcgtcgtcgtcttcttcttcctcctccttcgatctccacccatccaccgccggcgcggccgggatccgcagctcgagaccgacggtggagcgcggcggcgagcatcagcagccgacgacggcggaggaggaggatccgcgccgccgccgtcccgagacgccgccgccacccacg

SEQ ID NO: 48 - DNA, 890 bp, Sorghum bicolor
  misc_feature: Ceres Promoter Annt 8669907
ccgggaagatagccaactacccgtcgaaacaacaatttgcactgcatttccgatttctttattagttgaaaatagactattagtaaagaaccattttataactatgtaaaattagggtctatttagcgggtttttttttcaagtgctccttggccagagtacttgagaaaaagtcactttgtaaattcggttcatatcttaaaaatagcttcgactcctttactactcatcagagccttctaaagtgtttagttccgcttctttatggaagatgtcatagaaggtagagtcgtgctaaatagactttgaaaatactaaaaaggcattggtgaaaatatttttcttttcgatgataagtctattgtgatattgtgaaaactgtagacaatttatttgttttgtcaaataggattttgttaacgctgatgaaatcgcgatatcatatcataaatttgacaaagggtctacgtttttacatctactgagaccccttaaagtcctaccgagagaaacgtcgacaagacacgacatgggaagtacacaacgccattaccgcatatagtaccctccctctgctttcggctttgccatcaactgcaaccggacagcgaggtgaagtcaagttgccccgcggacgcaaagcatacaagtcatttgccgtttccggcgcgcgcctctgaacaaacccgaaagccccctcatcaccattcctcttcctgccatggcgatcccctttactcctcccctcccctcccaccaccaccactgcccctcccaactacaccacaccggcaccaccaccaaacgagcagctccaggctcttgctcaagaaggggagaagaggcgagcctttattgggaagttgctggaggagagaaggggaggaagg

SEQ ID NO: 49 - DNA, 1000 bp, Sorghum bicolor
  misc_feature: Ceres Promoter Annt 8642422
aatcagtctcagctgcaaacactccatacatgtgctaaatatatatagttgcatttagcaccccagcatctctggcacaaaaaagcaacgagtttccaaatatataacgtacgactaactatatattcaactattgtgcgagggtatttgcaacttattagactaatccttggattttctatcataagcgcgtacatttacaagggaagtgtgaatgaactgtaccatcattacctataagcctattccctattcattgttccatgtcacactctttaaacatatatatattgtcttctagagagcaaatgctgcttacaaattacaatgtatgatacaggcattgtttcttagaaatcaaacgtcaattaattgatgacagtggcctcgatcgctcataacatgtgaatgcggttttcaaaaattgcttcttgtgcctagatatatcttggatctcactctatgaagacactatagatatatagtatgctgagttggtgcacaaaagcatcagcaaatttttataatatgtattgcacctttatacaagaaacgtatctaataaattggagtcaaattaacatattttcatggtgtgctgacatttggagcagcaactagaactccagcttttgttgtatatatatgaaaactagatgtaaagcataagctaattatgtttgttgggataataccaacgaaacttccttcttaattagtgaattatcactgtatgcaagtgttttatttttaatttgccgtgcagcatttctatttttgtagttaattagctttcaccagatcatggcactgtggatgtccactaaacagaatttaatacaatccttacaaaattaattaaggatttttaaccctcagaattatatatttcttttgtcaaaagagcaaataaaaggagcatcaaggcgaaatcaaagaacaatcttattttttttctaaattctgccattgatcagtttgattgccttgtgccaacagc

SEQ ID NO: 50 - DNA, 20 bp, Artificial sequence
  UAS of HAP1 of Saccharomyces cerevisiae
agcacggacttatcggtcgg

SEQ ID NO: 51 - DNA, 22 bp, Artificial sequence
  UAS of LexA of Escherichia coli
gcagcggtattaacgggattac

SEQ ID NO: 52 - DNA, 20 bp, Artificial sequence
  UAS of Lac Operon of Escherichia coli
tactgtatatatatacagta

SEQ ID NO: 53 - DNA, 20 bp, Artificial sequence
  UAS of ArgR of Escherichia coli
aattgtgagcgctcacaatt

SEQ ID NO: 54 - DNA, 18 bp, Artificial sequence
  UAS of AraC of Escherichia coli
wntgaatwwwwattcanw

SEQ ID NO: 55 - DNA, 17 bp, Artificial sequence
  synthetic Zn sequence
tatggataaaaatgcta

(19) United States
(12) Patent Application Publication
(10) Pub. No.: US 2011/0283378 A1
(43) Pub. Date: Nov. 17, 2011

(54) SWITCHGRASS BIOLOGICAL CONTAINMENT

(76) Inventors: Peter N. Mascia, Thousand Oaks, CA (US); Mary Mascia, Thousand Oaks, CA (US); Michael F. Portereiko, Thousand Oaks, CA (US); David VanDinh Dang, Oak Park, CA (US)

(21) Appl. No.: 13/115,455

(22) Filed: May 25, 2011

Related U.S. Application Data
(63) Continuation-in-part of application No. PCT/US2009/065656, filed on Nov. 24, 2009.
(60) Provisional application No. 61/117,612, filed on Nov. 25, 2008.

(51) Int. Cl. A01H 5/10; A01H 1/02

(52) U.S. Cl. 800/260; 800/320

(57) ABSTRACT

The invention relates to materials and methods useful for controlling the unwanted spread of energy crop plants. The methods involve an F₁ hybrid transgenic switchgrass plant containing a transgene that affects a developmental stage such as spikelet meristem identity, establishment of floral meristem identity, or floral organ initiation, development, or function. The methods also involve one or more transcription factors that activate expression of the transgene. Such F₁ hybrid plants are incapable of forming viable seeds.

### CROSS-REFERENCE TO RELATED APPLICATIONS

This application claims priority to PCT/US2009/065656, filed Nov. 24, 2009, and U.S. Application Ser. No. 61/117,612, filed on Nov. 25, 2008. The disclosures of the prior applications are considered part of (and are incorporated by reference in) the disclosure of this application.

### STATEMENT AS TO FEDERALLY SPONSORED RESEARCH

Funding for the work described herein was provided by the federal government (U.S. Department of Agriculture Grant No. 8-3A75-6-501, program DE-PS36-06GO96002F), which has certain rights in the invention.

### TECHNICAL FIELD

The invention relates to methods and materials for biocontainment of transgenic plants. In particular, the invention pertains to methods and materials that can be used to minimize the unwanted transmission of transgenes in switchgrass.

### BACKGROUND

Switchgrass (Panicum virgatum) is a hardy, warm season perennial grass of the millet family. Switchgrass is native to the Central Plains of the United States and Canada and can grow up to 1.8 to 2.2 m in height. Switchgrass propagates by rhizomes and seeds produced on spikelets. A stand of switchgrass typically is not considered to reach its full potential until the third growing year. Switchgrass uses the C4 carbon fixation pathway which allows for improved water use efficiency during its growth period, providing an advantage under drought and high temperature conditions. Once established, switchgrass is also tolerant of flooding and grows rapidly, capturing a significant amount of solar energy and turning it into stored energy in the form of lignocellulosic components.

Switchgrass is used as pasture, as ground cover to control erosion and as a livestock feed. Switchgrass is highly effective in nitrogen fixation, and can be planted in crop rotation to replenish nutrients depleted from the soil by other crops such as corn.

Switchgrass can also be used as an energy crop. Switchgrass offers important advantages as an energy crop, in part because it can be liquified, gasified, or burned directly. Once established in a field, it typically is harvested annually or semiannually for 10 years or more before replanting. Ethanol production from switchgrass can provide as much as twenty times more net energy output than corn and removes considerably more CO₂ from the air. Switchgrass has the potential to produce up to 100 gallons (380 liters) of ethanol per metric ton of plant material, which gives switchgrass the potential to produce 1000 gallons of ethanol per acre, compared to 665 gallons of ethanol from sucrose from sugarcane and 400 gallons from the starch from corn.

Combustion of switchgrass pellets can result in only 3% to 4% of original mass remaining as ash due in part to switchgrass' lower silica and chloride content as compared to cool season grasses. Ash contents can be further reduced by allowing switchgrass to overwinter in the field, thereby reducing the silica and chloride contents further through the process of leaching. There are also advantages from an ash content perspective to producing switchgrass in sandy soils as opposed to clay soils, again based on silica and chloride contents.

Transgenic plants are now common in the agricultural industry. Desired transgenic traits in switchgrass include insect resistance, stress tolerance and increased biomass production. As transgenic switchgrass plants are developed and introduced into the environment, it is important to control the undesired spread of transgenic traits from transgenic switchgrass plants to other traditional and transgenic switchgrass varieties, or even other plant species. While physical isolation and pollen trapping border rows have been employed to control transgenic plants of other species under study conditions, these methods are cumbersome and are not practical for switchgrass. Effective ways to control the transmission and expression of transgenic traits without mechanical intervention would be useful for managing transgenic switchgrass plants used in biomass production.

### SUMMARY

The present disclosure features methods and materials useful for controlling the transmission of transgenic traits in switchgrass plants. The methods and materials of the invention minimize or even eliminate the undesired transmission of transgenic traits from one population of transgenic switchgrass plants to other populations of switchgrass plants and thus facilitate the cultivation of transgenic switchgrass.

In one aspect, the invention features a method for making switchgrass seed and F₁ seeds and plants produced by the method. The method includes crossing a plurality of first switchgrass plants grown in pollinating proximity to a plurality of second switchgrass plants. The first switchgrass plants are homozygous for a first exogenous nucleic acid, which comprises a transcription factor activation sequence operably linked to a plant sterility sequence. The second switchgrass plants are homozygous for a second exogenous nucleic acid, which comprises a regulatory region operably linked to a coding sequence for a transcription factor that binds to the activation sequence.

The method also includes collecting F₁ seeds formed on the first and/or the second switchgrass plants. F₁ switchgrass plants grown from the F₁ seeds express the plant sterility sequence and are sterile.

Either the first switchgrass plants, the second switchgrass plants, or both the first and second switchgrass plants are clonally propagated plants. For example, the first switchgrass plants can be clonally propagated plants whereas the second switchgrass plants are a genetically heterogeneous population of plants. Alternatively, both the first switchgrass plants and the second switchgrass plants can be clonally propagated plants. As another alternative, the first switchgrass plants can be a heterogeneous population of plants and the second switchgrass plants can be clonally propagated plants.

In some embodiments, the first switchgrass plants are clonally propagated tetraploid plants and exhibit an average self-compatibility percentage of less than 0.3%. In some embodiments, the first switchgrass plants are octaploid clonally propagated plants and exhibit a self-compatibility percentage of less than 1.3%. In some embodiments, the second switchgrass plants are tetraploid clonally propagated plants and exhibit an average self-compatibility percentage of less than 0.3%. In some embodiments, the second switchgrass plants are octaploid clonally propagated plants and exhibit a self-compatibility percentage of less than 1.3%.

In some embodiments, the F₁ seeds are collected from both the first and the second switchgrass plants. In some embodiments, the F₁ plants produce an average of less than 0.5 fertile seeds per plant. In some cases, the F₁ plants are incapable of producing male gametes, female gametes, or both male and female gametes.

The average crossability percentage between the first and the second switchgrass plants can be from about 50% to about 95%. For example, the first and the second switchgrass plants can be tetraploid, of the lowland ecotype, and have an average crossability percentage from about 80% to about 95%, e.g., from about 86% to about 91%.

The first switchgrass plants can exhibit a compact inflorescence and the second switchgrass plants exhibit a diffuse inflorescence. The first switchgrass plants can exhibit a uniform flowering time and the second switchgrass plants exhibit a non-uniform flowering time. The second switchgrass plants can exhibit a compact inflorescence and the first switchgrass plants exhibit a diffuse inflorescence. The second switchgrass plants can exhibit a uniform flowering time and the first switchgrass plants exhibit a non-uniform flowering time. The seeds collected from the first switchgrass plants can have a statistically significant increase in average seed weight relative to seeds collected from the second switchgrass plants. The seeds collected from the second switchgrass plants have a statistically significant increase in average seed weight relative to seeds collected from the first switchgrass plants.

In some embodiments, the growing step comprises growing the switchgrass plants at a ratio of greater than 4:1 of the first switchgrass plants:second switchgrass plants. The growing step can comprise growing the switchgrass plants at a ratio of greater than 4:1 of the second switchgrass plants:first switchgrass plants. The first and second switchgrass plants can be tetraploid plants. The first and the second switchgrass plants can be lowland type switchgrass plants.

The first switchgrass plants can exhibit homozygosity for an exogenous nucleic acid comprising the first transcription factor activation sequence operably linked to a second plant sterility sequence. The first switchgrass plants can exhibit homozygosity for an exogenous nucleic acid comprising a second transcription factor activation sequence operably linked to a second plant sterility sequence, and the second switchgrass plants exhibit homozygosity for an exogenous nucleic acid comprising a regulatory region operably linked to a coding sequence for a second transcription factor that binds to the second activation sequence. The first and/or the second switchgrass plants can further comprise a transgene (e.g., a transgene conferring herbicide resistance). The first and/or the second switchgrass plants exhibit homozygosity for the transgene.

The plant sterility sequence can encode a polypeptide. For example, the polypeptide can have an HMM bit score greater than about 175, wherein the HMM is based on the amino acid sequences depicted in FIG. 1, and wherein the plant has decreased fertility as compared a control plant that does not include the nucleic acid. The polypeptide can include an AP2 domain having at least 80% sequence identity to residues 134 to 185 of SEQ ID NO:5, a CMX-1 motif, and a CMX-2 motif. In some embodiments, the polypeptide includes an AP2 domain having at least 90% sequence identity to residues 134 to 185 of SEQ ID NO:5, a CMX-1 motif, and a CMX-2 motif. In some embodiments, the polypeptide includes an AP2 domain having at least 95% sequence identity to residues 134 to 185 of SEQ ID NO:5, a CMX-1 motif, and a CMX-2 motif. In some embodiments, the polypeptide includes an amino acid sequence with at least 85% sequence identity to a sequence selected from the group consisting of set forth in SEQ ID NOs:5, 6, 8, 10, 11, 13, 15, 17, 19, 21, 22, 24, 25, 26, 27, 28, 29, and 31. In some embodiments, a plant sterility polypeptide includes a DUF640 domain.

In some embodiments, the plant sterility sequence includes at least 50 contiguous nucleotides of any one of the nucleotide sequences set forth in SEQ ID NOs: 1, 2, 3, or 32 and is transcribed into a transcription product.

The transcription factor can be a chimeric transcription factor comprising a binding domain selected from the group consisting of Hap1, AraC, PDR3, LEU3, Lex A, Lac Operon, ArgR and Synthetic Zn-finger proteins. The transcription factor can be a chimeric transcription factor comprising an activation domain selected from the group consisting of VP16, C1 protein, ATMYB2, HAFL-1, ANT, ALM2, AvrXa10, Viviparous 1 (VP1), DOF, and RISBZ1 activation domain. The regulatory region is a broadly expressing promoter, e.g., a maize ubiquitin promoter. The regulatory region can be a photosynthetic tissue promoter.

Plants grown from the F₁ seeds can have a statistically significant increase in biomass in a second or subsequent growing season relative to control switchgrass plants that lack the first and the second exogenous nucleic acids.

Also featured are a plurality of F₁ hybrid transgenic switchgrass seeds, made by a process comprising growing a plurality of first switchgrass plants in pollinating proximity to a plurality of second switchgrass plants, crossing the first switchgrass plants and the second switchgrass plants, and collecting F₁ seeds formed on the first and/or the second switchgrass plants. The first switchgrass plants are homozygous for a first exogenous nucleic acid, which comprises a transcription factor activation sequence operably linked to a plant sterility sequence. The second switchgrass plants are homozygous for a second exogenous nucleic acid, which comprises a regulatory region operably linked to a coding sequence for a transcription factor that binds to the activation sequence. Either the first switchgrass plants, the second switchgrass plants, or both the first and second switchgrass plants are clonally propagated plants. F₁ switchgrass plants grown from the F₁ seeds express the plant sterility sequence and are sterile. The first switchgrass plants and the second switchgrass plants can have a crossability percentage of greater than about 50% (e.g., greater than about 65%).

Also featured is a method for making switchgrass seed. The method comprises crossing a plurality of first switchgrass plants grown in pollinating proximity to a plurality of second switchgrass plants, and collecting F₁ seeds formed on the first and/or the second switchgrass plants. The first plants are homozygous for a first exogenous nucleic acid, which comprises a transcription factor activation sequence operably linked to a plant sterility sequence. The plant sterility sequence contains at least 50 contiguous nucleotides of any one of the nucleotide sequences set forth in SEQ ID NOs: 1, 2, 3, or 32. The second plants are homozygous for a second exogenous nucleic acid comprising a regulatory region operably linked to a coding sequence for a transcription factor that binds to the activation sequence. F₁ switchgrass plants grown from the F₁ seeds express the plant sterility sequence and are sterile.

Also featured is a method of growing switchgrass. The method comprises growing F₁ hybrid switchgrass plants during a first growing season, and harvesting biomass from the switchgrass plants in a second or subsequent growing season. The F₁ plants are hemizygous for a first exogenous nucleic acid, which comprises a transcription factor activation sequence operably linked to a plant sterility sequence. The plant sterility sequence can encode a polypeptide. For example, the polypeptide can have an HMM bit score greater than about 175, wherein the HMM is based on the amino acid sequences depicted in FIG. 1, and wherein the plant has decreased fertility as compared a control plant that does not include the nucleic acid. The polypeptide can include an AP2 domain having at least 80% sequence identity to residues 134 to 185 of SEQ ID NO:5, a CMX-1 motif, and a CMX-2 motif. In some embodiments, the polypeptide includes an AP2 domain having at least 90% sequence identity to residues 134 to 185 of SEQ ID NO:5, a CMX-1 motif, and a CMX-2 motif. In some embodiments, the polypeptide includes an AP2 domain having at least 95% sequence identity to residues 134 to 185 of SEQ ID NO:5, a CMX-1 motif, and a CMX-2 motif. In some embodiments, the polypeptide includes an amino acid sequence with at least 85% sequence identity to a sequence selected from the group consisting of set forth in SEQ ID NOs:5, 6, 8, 10, 11, 13, 15, 17, 19, 21, 22, 24, 25, 26, 27, 28, 29, and 31. In some embodiments, the plant sterility polypeptide contains a DUF640 domain. In some embodiments, the plant sterility sequence contains at least 50 contiguous nucleotides of any one of the nucleotide sequences set forth in SEQ ID NOs: 1, 2, 3, or 32. The F₁ plants are also hemizygous for a second exogenous nucleic acid comprising a regulatory region operably linked to a coding sequence for a transcription factor that binds to the activation sequence. The F₁ switchgrass plants express the plant sterility sequence and are sterile.

This disclosure also features a plurality of F₁ transgenic switchgrass seeds. The seeds comprise a first exogenous nucleic acid comprising a transcription upstream activation sequence (UAS) and a first promoter, wherein the UAS and the first promoter are operably linked to a first sequence encoding a first plant sterility sequence, a second exogenous nucleic acid comprising the UAS and a second promoter, wherein the UAS and the second promoter are operably linked to a sequence encoding a second plant sterility sequence, wherein the first and the exogenous nucleic acids are different and affect a different developmental stage selected from the group consisting of i) spikelet meristem identity, ii) establishment of floral meristem identity, and iii) floral organ initiation, development, or function; and a third exogenous nucleic acid comprising a third promoter operably linked to a transcription factor, wherein the transcription factor binds the UAS, wherein F₁ switchgrass plants grown from the F₁ seeds express the plant sterility sequences and are sterile. The seeds can be hybrid seeds.

Also featured are a plurality of F₁ transgenic switchgrass seeds that include a first exogenous nucleic acid comprising a first transcription UAS and a first promoter, wherein the first UAS and the first promoter are operably linked to a sequence encoding a first plant sterility sequence, a second exogenous nucleic acid comprising a second UAS and a second promoter, wherein the second UAS and the second promoter are operably linked to a sequence encoding a second plant sterility sequence, wherein the first and the second exogenous nucleic acids are different and affect a different developmental stage selected from the group consisting of i) spikelet meristem identity, ii) establishment of floral meristem identity, and iii) floral organ initiation, development, or function; a third exogenous nucleic acid comprising a third promoter operably linked to a transcription factor, wherein the transcription factor binds the first UAS; and a fourth exogenous nucleic acid comprising a fourth promoter operably linked to a transcription factor, wherein the transcription factor binds the second UAS; wherein F₁ switchgrass plants grown from the F₁ seeds express the plant sterility sequences and are sterile. The seeds can be hybrid seeds.

In the F₁ transgenic switchgrass seeds described herein, at least one of the plant sterility sequences can encode a cytotoxic gene product such as a barnase polypeptide. The first and second nucleic acids can be a single nucleic acid molecule. The first or second plant sterility sequence can be an antisense nucleic acid or a ribozyme. The first or second plant sterility sequence can inhibit expression of a gene by post-transcriptional gene silencing (e.g., the plant sterility sequence can be a small interfering RNA). The transcription factor can be a chimeric transcription factor. For example, the chimeric transcription factor can include a binding domain selected from the group consisting of Hap1, LexA, Lac Operon, ArgR, AraC, PDR3, and LEU3 binding domain. A chimeric transcription factor can include an activation domain selected from the group consisting of VP16, C1 protein, ATMYB2, HAFL-1, ANT, ALM2, AvrXa10, Viviparous 1 (VP1), DOF, and RISBZ1 activation domain.

In the F1 transgenic switchgrass seeds described herein, the first or second plant sterility sequence can affect spikelet meristem identity and reduce expression of a polypeptide selected from the group consisting of IDS1, SID1, PAP2, SNB, LHS1, APO1, FZP, BD1, and IFA1. The first or second promoter can be selected from the group consisting of PD3796 (SEQ ID NO:40) or PD3800 (SEQ ID NO:41).

In the F₁ transgenic switchgrass seeds described herein, the first or second plant sterility sequence can affect establishment of floral meristem identity and reduce expression of a polypeptide selected from the group consisting of LHS1, AP1, CAL, LFY, and FUL. The first or second promoter can be selected from the group consisting of CeresAnnot:8643934 (SEQ ID NO:42); CeresAnnot:8632648 (SEQ ID NO: 43); CeresAnnot:8681303 (SEQ ID NO: 44); and CeresAnnot:8642422 (SEQ ID NO: 45).

In the F₁ transgenic switchgrass seeds described herein, the first or second plant sterility sequence can affect floral organ initiation, development, or function and reduce expression of a polypeptide selected from the group consisting of AP1, AP2, OsMADS3, MADS58, PI, AP3, SUPERWOMAN1, and AG. The first or second plant sterility sequence can affect floral organ initiation, development, or function and reduce expression of SHP1, SHP2, ANT, and CRC. The first or second promoter can be selected from the group consisting of CeresAnnot:8657974 (SEQ ID NO:46); CeresAnnot:8732691 (SEQ ID NO:47); CeresAnnot:8031970 (SEQ ID NO:48); and CeresAnnot:8669907 (SEQ ID NO:49).

In the F₁ transgenic switchgrass seeds described herein, the first plant sterility sequence can reduce expression of a nucleic acid having at least 80% identity to a nucleotide sequence selected from the group consisting of SEQ ID NO: 33, 34, 35, and 36. The first promoter can be selected from the group consisting of PD3796 (SEQ ID NO:40) or PD3800 (SEQ ID NO:41).

In the F₁ transgenic switchgrass seeds described herein, the second plant sterility sequence can reduce expression of a nucleic acid having at least 80% identity to a nucleotide sequence set forth in SEQ ID NO:36 or SEQ ID NO:37, wherein if the first sterility sequence reduces expression of the nucleic acid having at least 80% identity to SEQ ID NO:36, the second plant sterility sequence reduces expression of the nucleic acid having at least 80% identity to SEQ ID NO:37. A second promoter can be selected from the group consisting of CeresAnnot:8643934 (SEQ ID NO:42); CeresAnnot:8632648 (SEQ ID NO: 43); CeresAnnot:8681303 (SEQ ID NO:44); and CeresAnnot:8642422 (SEQ ID NO:45).

In the F₁ transgenic switchgrass seeds described herein, the second plant sterility sequence reduces expression of a nucleic acid having at least 80% identity to a nucleotide sequence selected from the group consisting of SEQ ID NO:37, 38, and 39. A second promoter can be selected from the group consisting of CeresAnnot:8657974 (SEQ ID NO:46); CeresAnnot:8732691 (SEQ ID NO:47); CeresAnnot:8031970 (SEQ ID NO:48); and CeresAnnot:8669907 (SEQ ID NO:49).

In the F₁ transgenic switchgrass seeds described herein, the first plant sterility sequence can reduce expression of a nucleic acid having at least 80% identity to a nucleotide sequence set forth in SEQ ID NO:36 or SEQ ID NO:37. The first promoter can be selected from the group consisting of CeresAnnot:8643934 (SEQ ID NO:42); CeresAnnot:8632648 (SEQ ID NO: 43); CeresAnnot:8681303 (SEQ ID NO:44); and CeresAnnot:8642422 (SEQ ID NO:45). The second plant sterility sequence can reduce expression of a nucleic acid having at least 80% identity to a nucleotide sequence selected from the group consisting of SEQ ID NO:37, 38, and 39, wherein if the first gene product reduces expression of the nucleic acid having at least 80% identity to SEQ ID NO:37, the second gene product reduces expression of the nucleic acid having at least 80% identity to SEQ ID NO:38 or SEQ ID NO:39. A second promoter can be selected from the group consisting of CeresAnnot:8657974 (SEQ ID NO:46); CeresAnnot:8732691 (SEQ ID NO:47); CeresAnnot:8031970 (SEQ ID NO:48); and CeresAnnot:8669907 (SEQ ID NO:49).

This disclosure also features a method for making switchgrass seed. The method includes crossing a plurality of first switchgrass plants grown in pollinating proximity to a plurality of second switchgrass plants, and collecting F₁ seeds formed on the first and/or the second switchgrass plants, wherein F₁ switchgrass plants grown from the F₁ seeds express the plant sterility sequences and are sterile. The F₁ plants can produce an average of less than 0.5 fertile seeds per panicle. In one embodiment, the first plants comprise a first exogenous nucleic acid comprising a transcription UAS and a first promoter, wherein the UAS and the first promoter are operably linked to a first sequence encoding a first plant sterility sequence, and a second exogenous nucleic acid comprising the UAS and a second promoter, wherein the UAS and the second promoter are operably linked to a sequence encoding a second plant sterility sequence, wherein the first and the second exogenous nucleic acids are different and affect a different developmental stage selected from the group consisting of iii) spikelet meristem identity, iv) establishment of floral meristem identity, and v) floral organ initiation, development, or function, wherein the first switchgrass plants are homozygous for the first and second exogenous nucleic acids. In such an embodiment, the second plants comprise a third exogenous nucleic acid comprising a third promoter operably linked to a transcription factor, wherein the transcription factor binds the UAS, wherein the second switchgrass plants are homozygous for the third exogenous nucleic acid.

In one embodiment, the first plants comprise a first exogenous nucleic acid comprising a transcription UAS and a first promoter, wherein the UAS and the first promoter are operably linked to a first sequence encoding a first plant sterility sequence, and a second exogenous nucleic acid comprising the UAS and a second promoter, wherein the UAS and the second promoter are operably linked to a sequence encoding a second plant sterility sequence, wherein the first and the second exogenous nucleic acids are different and affect a different developmental stage selected from the group consisting of iii) spikelet meristem identity, iv) establishment of floral meristem identity, and v) floral organ initiation, development, or function, wherein the first switchgrass plants are homozygous for the first and second exogenous nucleic acids. In such an embodiment, the second plants can include a third exogenous nucleic acid comprising a third promoter operably linked to a transcription factor, wherein the transcription factor binds the first UAS; and a fourth exogenous nucleic acid comprising a fourth promoter operably linked to a transcription factor, wherein the transcription factor binds the second UAS, wherein the second switchgrass plants are homozygous for the third and fourth exogenous nucleic acids.

Also featured is a method of growing switchgrass. The method includes growing F₁ switchgrass plants for at least one growing season, the plants comprising a first exogenous nucleic acid comprising a transcription UAS and a first promoter, wherein the UAS and the first promoter are operably linked to a first sequence encoding a first plant sterility sequence, a second exogenous nucleic acid comprising the UAS and a second promoter, wherein the UAS and the second promoter are operably linked to a sequence encoding a second plant sterility sequence, and a third exogenous nucleic acid comprising a third promoter operably linked to a transcription factor, wherein the transcription factor binds the UAS, wherein the first and the second exogenous nucleic acids are different and affect a different developmental stage selected from the group consisting of iv) spikelet meristem identity, v) establishment of floral meristem identity, and vi) floral organ initiation, development, or function; and wherein the switchgrass plants are hemizygous for the first, second, and third exogenous nucleic acids; and harvesting biomass from the switchgrass plants in a second or subsequent growing season.

In another aspect, a method of growing switchgrass can include growing F₁ switchgrass plants for at least one growing season, the plants comprising a first exogenous nucleic acid comprising a transcription UAS and a first promoter, wherein the UAS and the first promoter are operably linked to a first sequence encoding a first plant sterility sequence, a second exogenous nucleic acid comprising the UAS and a second promoter, wherein the UAS and the second promoter are operably linked to a sequence encoding a second plant sterility sequence, a third exogenous nucleic acid comprising a third promoter operably linked to a transcription factor, wherein the transcription factor binds the first UAS; and a fourth exogenous nucleic acid comprising a fourth promoter operably linked to a transcription factor, wherein the transcription factor binds the second UAS, wherein the first and second exogenous nucleic acids are different and affect a different developmental stage selected from the group consisting of v) spikelet meristem identity, vi) establishment of floral meristem identity, and vii) floral organ initiation, development, or function; wherein the switchgrass plants are hemizygous for the first, second, third, and fourth exogenous nucleic acids; and harvesting biomass from the switchgrass plants in a second or subsequent growing season.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. In some instances, features of the invention may consist essentially of that feature rather than comprise that feature. Section headings are provided merely for convenience. The word “comprising” in the claims may be replaced by “consisting essentially of” or with “consisting of,” according to standard practice in patent law.

Other features and advantages of the invention will be apparent from the following detailed description.

### DESCRIPTION OF THE DRAWING

FIG. 1 (A-E) is an alignment of the amino acid sequence corresponding to Ceres Clone: 123905 (SEQ ID NO:5) with homologous and/or orthologous amino acid sequences. In this alignment, a dash in an aligned sequence represents a gap, i.e., a lack of an amino acid at that position. Identical amino acids or conserved amino acid substitutions among aligned sequences are identified by boxes. FIG. 1 was generated using the program MUSCLE version 3.52.

### DETAILED DESCRIPTION

This disclosure provides methods and materials for effectively minimizing the unwanted transmission of recombinant DNA from transgenic switchgrass plants to other switchgrass populations. The disclosure is based, in part, on the discovery that developmentally appropriate expression of certain nucleic acid constructs can successfully control fertility in transgenic switchgrass, despite the fact that switchgrass has different ploidy levels and exhibits significant self-incompatibility. The methods described herein result in the production of sterile switchgrass plants that can be grown on a commercial scale with less concern about unwanted spread of transgenes present in such plants. Furthermore, sterility in switchgrass is such that it can be easily scored in the field, which helps in assessing transgene effect and allows remedial actions, if necessary, to be taken. Easy visual assessment also helps in breeding new varieties most likely to show the sterility outcome.

As described herein, developmentally appropriate expression of a sterility polypeptide such as the polypeptide set forth in SEQ ID NO:5 or a homolog thereof, can cause an anthesis defect in switchgrass. The anthesis defect is readily apparent as expression of such plant sterility polypeptides can prevent emergence of the orange colored anthers from the florets. The presence or absence of orange-colored anthers can easily be observed in a field without a need for more sophisticated or more time-consuming assays. Furthermore, within the few open florets in the switchgrass, seed set may be reduced.

In addition, transgenic switchgrasses described herein can express two or more different plant sterility sequences that affect different developmental stages such as establishment of spikelet meristem identity, establishment of floral meristem identity, or floral organ initiation, development, or function, resulting in a visible abnormality at the specified stage and in some cases, subsequent stages, which negatively influence normal reproductive development of the plant. See, for example, Thompson and Hake, Plant Phys., 149:38-45 (2009), for a review of the developmental stages in grass. Such transgenic plants are sterile.

Sterility caused by the polypeptide set forth in SEQ ID NO:5 or a homolog thereof, or by reduced expression of polypeptides encoded by the nucleic acids of SEQ ID NOs:33-39, does not cause biomass yield drag and is such that panicle formation still occurs in a way that does not alter panicle contribution to the biomass yield component. In contrast, some other sterility polypeptides act by a mechanism that impairs panicle growth or diminishes plant growth.

### I. DEFINITIONS

“Cell type-preferential promoter” or “tissue-preferential promoter” refers to a promoter that drives expression preferentially in a target cell type or tissue, respectively, but may also lead to some transcription in other cell types or tissues as well.

“Control plant” refers to a switchgrass plant that does not contain the exogenous nucleic acid present in a transgenic plant of interest, but otherwise has the same or similar genetic background as such a transgenic plant. A suitable control plant can be a non-transgenic wild type plant, a non-transgenic segregant from a transformation experiment, or a transgenic plant that contains an exogenous nucleic acid other than the exogenous nucleic acid of interest.

“Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

“Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Expression” refers to the process of converting genetic information of a polynucleotide into RNA through transcription, which is catalyzed by an enzyme, RNA polymerase, and into protein, through translation of mRNA on ribosomes.

“Heterologous polypeptide” as used herein refers to a polypeptide that is not a naturally occurring polypeptide in a switchgrass plant cell, e.g., a transgenic Panicum virgatum plant transformed with and expressing the coding sequence for a nitrogen transporter polypeptide from a Zea mays plant.

“Nucleic acid” and “polynucleotide” are used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers.

“Operably linked” refers to the positioning of a regulatory region and a sequence to be transcribed in a nucleic acid so that the regulatory region is effective for regulating transcription or translation of the sequence. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site.

“Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

“Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation sequence (UAS). For example, a suitable enhancer is a cis-regulatory element (−212 to −154) from the upstream region of the octopine synthase (ocs) gene. Fromm et al., The Plant Cell, 1:977-984 (1989).

“Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

### II. METHODS FOR MAKING STERILE SWITCHGRASS

In one aspect, the invention features methods for making sterile F₁ hybrid switchgrass seeds and plants. The methods involve crossing a plurality of first switchgrass plants with a plurality of second switchgrass plants. Each of the two types of parent plants contain one or more transgenes that, when combined in the F₁ progeny, operate in combination such that the F₁ progeny seeds can germinate while the F₁ plants grown from such seeds are sterile.

As explained in more detail below, the first switchgrass plants contain a first nucleic acid construct that comprises a transcription factor UAS and promoter that are operably linked to a plant sterility sequence. The second switchgrass plants contain a nucleic acid encoding a transcription factor that is effective for binding to the UAS.

In some embodiments, the first switchgrass plants contain at least one nucleic acid construct that comprises a) a first transcription factor UAS and a first promoter that are operably linked to a first plant sterility sequence and b) a second transcription factor UAS and a second promoter that are operably linked to a second plant sterility sequence. The second switchgrass plants contain a nucleic acid encoding a transcription factor that is effective for binding to the first UAS and a nucleic acid encoding a transcription factor that is effective for binding to the second UAS. Alternatively, the first switchgrass plants can contain at least one nucleic acid construct that comprises a) a first transcription factor UAS and a first promoter that are operably linked to a first plant sterility sequence and b) a nucleic acid encoding a transcription factor that is effective for binding to a second UAS. The second switchgrass plants can contain at least one nucleic acid construct that comprises a) a second transcription factor UAS and a second promoter that are operably linked to a second plant sterility sequence and b) a nucleic acid encoding a transcription factor that is effective for binding to the first UAS.

In some embodiments, a single transcription factor activates both plant sterility sequences, each of which is operably linked to the same upstream activation sequence. Alternatively, two different transcription factors can be expressed such that each of the transcription factors activates one of the plant sterility sequences. Each sterility sequence can have a different expression pattern such that different developmental stages (e.g., establishment of spikelet meristem identity, establishment of floral meristem identity, or floral organ initiation, development, or function) can be impacted.

Upon crossing of the two types of switchgrass plants, seed development ensues. Expression of the transcription factor, either in F₁ seeds or F₁ plants, activates transcription of the plant sterility sequence, which in turn results in the F₁ plants being sterile. Transfer of these transgenes, or any other transgene(s) present in such plants, to other switchgrass plants is minimized or eliminated because all, or substantially all, of the F₁ plants are sterile. Thus, unwanted spread of transgenes to other switchgrass plants is effectively prevented.

Parent Plants

There are two different general switchgrass ecotypes, lowland and upland. Lowland switchgrass are predominantly tetraploid (2n=4x=36 chromosomes) while upland switchgrass cultivars are predominantly octaploid (2n=8x=72 chromosomes). Transgenic switchgrass plants to be used as parents can be crossed with other parent transgenic switchgrass plants that are of the same ecotype, as well as plants of another ecotype that have the same ploidy level.

Typically, either the first and/or the second switchgrass parent plants are clonally propagated plants. A particularly useful technique for producing clonally propagated first and/or second switchgrass parents is described in Application No. PCT/US2009/051355, filed Jul. 22, 2009. The first switchgrass parent plant, the second switchgrass parent plant, or both parents, can serve as the female parent in such methods. Clonally propagated switchgrass plants exhibit heterozygosity at many loci but, because each plant is produced by propagation from the same clone, each plant has substantially the same genotype. Thus the clonally propagated plants used as parents can be considered to be genetically uniform. It will be appreciated that clonally propagated parent plants may have a minor proportion of non-clonally propagated plants, either deliberately added or inadvertently present.

In some embodiments, the first plants are clonally propagated plants, while the second plants are of a switchgrass variety or line that has not been clonally propagated and thus is genetically heterogeneous. Conversely, the first plants can be clonally propagated plants, while the second plants can be of a switchgrass variety or line that has not been clonally propagated. Having one type of parent plant that is genetically heterogeneous can maintain genetic diversity in the sterile F₁ progeny so that the F₁ plants can adapt to diverse environmental conditions that may occur during the years that the stand of F₁ plants is used for commercial purposes. Either the first or the second switchgrass parent plants can serve as the female parent in these embodiments.

A switchgrass variety or line suitable for use as one of the parents in the methods described herein can be developed by plant breeding procedures generally described in, e.g., Allard, Principles of Plant Breeding, John Wiley & Sons, Inc. (1960); Simmonds, Principles of Crop Improvement, Longman Group Limited (1979); and, Jensen, Plant Breeding Methodology, John Wiley & Sons, Inc. (1988). Detailed breeding methodologies specifically applicable to switchgrass take into account the necessity of reaching homozygosity for the transgene(s) that are to be present in the parent plants. For example, a switchgrass variety can be developed by a program of mass selection. In mass selection, desirable individual plants are chosen, harvested, and the seed composited without progeny testing to produce the next generation. Since selection is based on the maternal parent only, and there is no control over pollination, mass selection amounts to a form of random mating with selection. Mass selection typically increases the proportion of desired genotypes in the population. Alternatively, a program of selection with progeny testing can be utilized. A program of selection with progeny testing is generally preferred over mass selection. Examples of selection with progeny testing breeding programs for switchgrass include Restricted Recurrent Phenotypic Selection (RRPS) and Between and Within Half-Sib Family Selection (B&WFS) for varietal improvement. Switchgrass varieties suitable as parents can be developed by either of these programs. Another alternative is to develop switchgrass parent varieties in a Genotypic Recurrent Selection program. Taliaferro, Breeding and Selection of New Switchgrass Varieties for Increased Biomass Production, Oak Ridge National Laboratory USA (2002). Genotypic Recurrent Selection relies on analysis of half-sib progeny performance in the year following the establishment year. As another alternative, a synthetic variety can be developed for use as a parent. A synthetic variety is produced by crossing several initial source plants. The number of initial plant varieties, populations, wild accessions, ecotypes, etc., that are used to develop a synthetic can vary from as little as 10 to as much as 500. Typically, about 100 to 300 varieties, populations, etc., are used to initiate development of the synthetic variety. Seed from the initial seed production plot can subsequently undergo one or more generations of multiplication, depending on the number of generations needed to reach homozygosity for the transgene(s) and the amount of seed desired for performing the parental cross.

Transgenic switchgrass plants can be entered into a breeding program to introduce a different exogenous nucleic acid into the switchgrass line or for further selection of other desirable traits, before using the plants as parents to make F₁ hybrids.

Transgene Inheritance

Regardless of whether or not the parent plants are obtained by clonal propagation, switchgrass plants that are to be used as parents in methods described herein are bred to exhibit homozygosity for the transgene(s) involved in conferring plant sterility. Switchgrass is an allotetraploid or allooctaploid and, thus, generally exhibits disomic inheritance for a given genetic locus, including a transgene locus. However, not all loci will follow a simple inheritance pattern because preferential pairing between homologous chromosomes and double reduction may occasionally occur in switchgrass, leading to segregation distortion in some instances.

Therefore, it is generally desirable to confirm that a particular transgenic event behaves as a homozygote before proceeding to use plants from that event as parents in the methods. Thus, for example, transgenic switchgrass plants containing a first exogenous nucleic acid (comprising one or more plant sterility sequences) are selected to be homozygous and exhibit simple Mendelian inheritance for the exogenous nucleic acid. As another example, transgenic switchgrass plants containing a second exogenous nucleic acid (comprising one or more transcription factor coding sequences) are selected to be homozygous and exhibit simple Mendelian inheritance for the exogenous nucleic acid. As another example, transgenic switchgrass plants containing a third exogenous nucleic acid (comprising a sequence of interest) are selected to be homozygous and exhibit simple Mendelian inheritance for the exogenous nucleic acid. In this regard, progeny testing via molecular analysis can be particularly useful during backcrossing to obtain a population that contains the exogenous nucleic acid. Polycross sib mating of the population followed by progeny testing to identify homozygous individuals can then yield the desired transgenic parent line.

Crossing Parent Plants

The first and second switchgrass parent plants are crossed by growing a plurality of the two types of plants in pollinating proximity. The two types of parent plant can be planted in separate rows or can be randomly interplanted, and grown in a field under agronomic practices suitable for switchgrass and known in the art. In either scheme, the ratio of first parent plants to second parent plants can vary from 1:10 to 10:1, e.g., the first parent:second parent ratio can be 9:1, 4:1, 1:1, 1:4, or 1:9. The choice of a suitable ratio can be made by one of ordinary skill based on factors such as pollen shed of the male parent and pollen receptivity of the female parent.

Crossing typically occurs via wind pollination, although can also occur via manual pollination, e.g., plants of first type can be pollinated by hand with pollen from plants of the second type, and/or plants of the second type can be pollinated by hand with pollen from plants of the first type. In some embodiments, pollination involves removing pollen-forming structures on plants one set of parent plants in order to prevent self-pollination, thereby permitting manual or natural pollination by pollen from the other set of plants.

Switchgrass exhibits partial or complete self-incompatibility. Thus, both the first and the second switchgrass plants can serve as the female parents in the methods, each type of plant fertilized by pollen from the other parent. It is sometimes desirable have seeds preferentially formed on only one of the parents. In such cases, the parent on which seeds preferentially form is termed a pseudo female and the parent that serves as the pollen donor is termed a pseudo male.

When complete self-incompatibility is present, switchgrass plants used as parents in the methods described herein do not require measures such as male sterility systems or removal of pollen-forming structures in order for cross-pollination to occur. For tetraploid switchgrass plants, complete self-incompatibility refers to an average self-compatibility percentage of less than 0.3%, as determined by the method of Martinez-Reyna et al. Crop Sci. 42:1800-1805 (2002). For octaploid switchgrass plants, complete self-incompatibility refers to an average self-compatibility percentage of less than 1.3%, also determined by the method of Martinez-Reyna et al. Crop Sci. 42:1800-1805 (2002). Using parents that are completely self-incompatible ensures that the seed produced in a production field is primarily or even exclusively F₁ hybrid seed.

It is desirable to use parents that have been demonstrated to produce a high percentage of progeny seed, measured by crossability percentage. Crossability percentage refers to the percentage of seeds obtained per floret emasculated and fertilized after controlled crosses between plants of two different switchgrass varieties or populations as described in Martinez-Reyna et al. Crop Sci. (38:876-878 (1998) and Martinez-Reyna et al. Crop Sci. 42:1800-1805 (2002). Thus, it is desirable to use parents whose crossability percentage is greater than 50%, e.g., 50% to 65%, 55% to 65%, 60% to 70%, 66% to 85%, 66% to 80%, 69% to 85%, 69% to 95%, 70% to 75%, 73% to 80%, 75% to 95%, 80% to 95%, 85% to 95%, 85% to 90%, 80% to 90%, 90% to 95%, or any range between 66% and 95%. Crossability percentage is influence by factors such as whether or not the parents flower at a similar time. Furthermore, not all pairs will necessarily result in sterile offspring due to, for example, the effect that the genome position where a transgene is inserted may have on self-incompatibility. Therefore, candidate parent pairs are typically crossed in pairwise combination in order to identify those parent pairs that have a suitable crossability percentage.

If one or both parents have partial self-incompatibility (average self-compatibility percentages of 0.3% or more for tetraploids and 1.3% or more for octaploids), plants of the first type can be pollinated by hand with pollen from plants of the second type, and/or plants of the second type can be pollinated by hand with pollen from plants of the first type. In some embodiments, pollen-forming structures on plants of the first type are removed in order to prevent self-pollination, thereby permitting manual or wind pollination by pollen from second plants.

In some embodiments, one type of parent plant exhibits a compact inflorescence. The other type may exhibit a diffuse inflorescence. The parent having a compact inflorescence in such embodiments will have less shattering and, when such a parent is the female, serves to increase the yield of F₁ hybrid seed obtained from the cross.

In some embodiments, one type of parent plant exhibits a uniform flowering time. The other type may exhibit a non-uniform flowering time. The parent having a uniform flowering time in such embodiments will have a more uniform harvest period and, when such a parent is the female, serves to facilitate harvesting operations when collecting the F₁ hybrid seed.

Collecting Seed

Seed maturation in switchgrass typically occurs over approximately a one month period following fertilization. The F₁ seeds are collected once the appropriate stage of seed development has been reached, either by harvesting seeds from one of the parent plants (the type intended to served as the female parent) or by harvesting seeds from both types of parent plants. Either technique of harvesting is encompassed by the methods described herein. If F₁ seeds are collected from only one parental type, the female plants are preferably plants that have a compact inflorescence and/or a uniform flowering time. The presence of one or both traits in females can minimize the effect of seed shattering, which reduces the yield of F₁ seeds. The presence of a uniform flowering trait will also serve to minimize the amount of time required to harvest seeds.

F₁ hybrid seeds produced by the methods described herein are sterile, i.e., such seeds have a high germination percentage, but the resulting F₁ hybrid plants produce little or no F₂ seeds. The germination percentage for such F₁ seed is greater than 80%, as determined on unsized seed by the method of Aiken et al., J. Range Management 48: 455-458 (1995), e.g., greater than 85%, 86%, 87%, 88%, 89%, or 90%. F₁ plants are considered to be sterile when the average number of F₂ seed produced by such F₁ plants is less than 0.5 viable seeds per panicle, e.g., less than 0.4, 0.3, 0.2, 0.1, 0.05, 0.01, or 0.005 fertile seeds per panicle. F₁ plants are also considered to be sterile when the average number of F₂ seeds is so low as to be undetectable. The average number of F₂ seeds per plant is calculated by isolating seeds as described in Crop Sci. 47: 636-642 (2007) from at least 100 F₁ plants, determining the number of seeds that germinate by the procedure of Aiken et al. 1995, supra, and dividing the number of germinating seeds by the number of F₁ plants.

In some embodiments, F₁ seeds collected from one type of parent switchgrass plants have a statistically significant increase in the average weight per 100 seeds relative to the average weight per 100 F₁ seeds collected from the other type of parent plants. Average weight per 100 seeds is determined by standard methods, and typically ranges from about 50 mg to about 160 mg/100 seeds for lowland ecotypes, e.g., 60, 70, 80, 90, 100, 110, 120, 130, 140, 150, or 160 mg per 100 seeds. Thus, for example, one type of lowland parent plant may produce seeds having an average weight per 100 seeds of from about 80 to about 100, or about 100 to about 120, or about 120 to about 160 mg per 100 seeds, and that is significantly higher than the average for the other type of parent plant. Average weight per 100 seeds typically ranges from about 100 mg to about 230 mg/100 seeds for upland ecotypes, e.g., 100, 110, 120, 130, 140, 150, 160, 170, 180, 190, 200, 210, 220, 230, 240, 250, or 260 mg per 100 seeds. For example, one type of upland parent plant may produce seeds having an average weight per 100 seeds of from about 100 to about 120, or about 120 to about 160, or about 160 to about 180, or about 180 to about 200, or about 200 to about 220, or about 220 to about 240, or about 240 to about 160 mg per 100 seeds, and that is significantly higher than the average for the other type of parent plant.

Typically, a difference in the amount of a parameter relative to a control is considered statistically significant at p≦0.05 with an appropriate parametric or non-parametric statistic, e.g., Chi-square test, Student's t-test, Mann-Whitney test, or F-test. Thus, for example, a higher average weight per 100 seeds for F₁ seeds from one type of parent plant relative to the average weight per 100 seeds for the other type of parent plant is considered statistically significant at p<0.01, p<0.005, or p<0.001.

### III. NUCLEIC ACIDS

Plant Sterility Sequences. F₁ transgenic switchgrass plants described herein contain an exogenous nucleic acid comprising a plant sterility sequence operably linked to a transcription factor UAS. Overexpression or timely expression of a plant sterility sequence, which is controlled by the UAS, results in the production of F₁ seeds that have a high germination percentage and F₁ plants that are sterile, e.g., that produce no or abnormal floral structures, or produce floral structures that cannot form male and/or female gametes. One of ordinary skill in the art will appreciate that the term “plant sterility sequence” refers to the plant sterility effect and is not limited to plant sequences. As described herein, a plant sterility sequence can affect establishment of spikelet meristem identity, establishment of floral meristem identity, or floral organ initiation, development, or function.

In some embodiments, a plant sterility sequence encodes a polypeptide that contains an AP2 domain. The AP2 domain is found in transcription factor proteins and can bind DNA. The AP2 family of transcription factors can include a nuclear localization domain and an activation domain. The AP2 family of transcription factors also can include a CMX-1 motif (EXEX₄VX₂LX₂VXSGX₅P) and a CMX-2 motif (CX₂CX₄CX₂₋₄C). The CMX-2 motif is a putative zinc-finger motif that may be involved in DNA binding or in protein-protein interactions. See, Nakano et al., Plant Physiol., 140:411-432 (2006). In some embodiments, a polypeptide can include a variant of the CMX-1 motif. Such variants differ from the CMX-1 motif by one, two, or three amino acid substitutions.

SEQ ID NO:5 sets forth the amino acid sequence of an Arabidopsis thaliana clone, identified herein as Ceres Clone Id No. 123905 (SEQ ID NO:5), that is predicted to encode a polypeptide containing an AP2 domain, a CMX-1 motif, and a CMX-2 motif. Overexpression of SEQ ID NO:5 or homologs thereof affects establishment of floral meristem identity, or floral organ initiation, development, or function. A plant sterility sequence can encode a polypeptide that includes an AP2 domain having 70 percent or greater (e.g., 75%, 80%, 85%, 90%, 95%, 97%, 98%, 99%, or 100%) sequence identity to residues 134 to 185 of SEQ ID NO:5. In some embodiments, a plant sterility sequence encodes a polypeptide containing an AP2 domain having 70 percent or greater (e.g., 75%, 80%, 85%, 90%, 95%, 97%, 98%, 99%, or 100%) sequence identity to the AP2 domain of one or more of the polypeptides set forth in SEQ ID NOs: 6, 8, 10, 11, 13, 15, 17, 19, 21, 22, 24, 25, 26, 27, 28, 29, and 31. For example, a plant sterility sequence can encode a polypeptide having 70 percent or greater sequence identity to residues 95 to 146 of SEQ ID NO:6, residues 116 to 167 of SEQ ID NO:8, residues 125 to 176 of SEQ ID NO:10, residues 130 to 181 of SEQ ID NO:11, residues 137 to 188 of SEQ ID NO:13, residues 143 to 194 of SEQ ID NO:15, residues 127 to 178 of SEQ ID NO:17, residues 131 to 182 of SEQ ID NO:19, residues 135 to 186 of SEQ ID NO:21, residues 120 to 171 of SEQ ID NO:22, residues 128 to 179 of SEQ ID NO:24, residues 133 to 184 of SEQ ID NO:25, residues 135 to 186 of SEQ ID NO:26, residues 121 to 172 of SEQ ID NO:27, residues 153 to 204 of SEQ ID NO:28, residues 118 to 169 of SEQ ID NO:29, or residues 130 to 181 of SEQ ID NO:31. The polypeptides set forth in SEQ ID NOs: 8, 11, 13, 15, 17, 19, 21, 22, 24, 25, and 26 also contain CMX-1 and CMX-2 motifs as set forth in the Sequence Listing. The polypeptides set forth in SEQ ID NOs: 6, 10, 27, 28, 29, and 31 also contain variants of the CMX-1 motif and contain CMX-2 motifs as set forth in the Sequence Listing.

“Percent sequence identity” refers to the degree of sequence identity between any given reference sequence, e.g., SEQ ID NO:5 or portion thereof such as an AP2 domain, and a candidate plant sterility sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chema et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw). To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some embodiments, one or more functional homologs of a reference plant sterility polypeptide containing an AP2 domain, and preferably a CMX-1 motif and/or a CMX-2 motif can be used in the methods described herein. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a plant sterility polypeptide, or by combining domains from the coding sequences for different naturally-occurring plant sterility polypeptides (“domain swapping”). The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of plant sterility polypeptides. Sequence analysis can involve BLAST, Reciprocal BLAST, or PSI-BLAST analysis of nonredundant databases using a plant sterility polypeptide amino acid sequence as the reference sequence. Amino acid sequence is, in some instances, deduced from the nucleotide sequence. Those polypeptides in the database that have greater than 40% sequence identity are candidates for further evaluation for suitability as a plant sterility polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in plant sterility polypeptides, e.g., conserved functional domains.

Conserved regions can be identified by locating a region within the primary amino acid sequence of a plant sterility polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

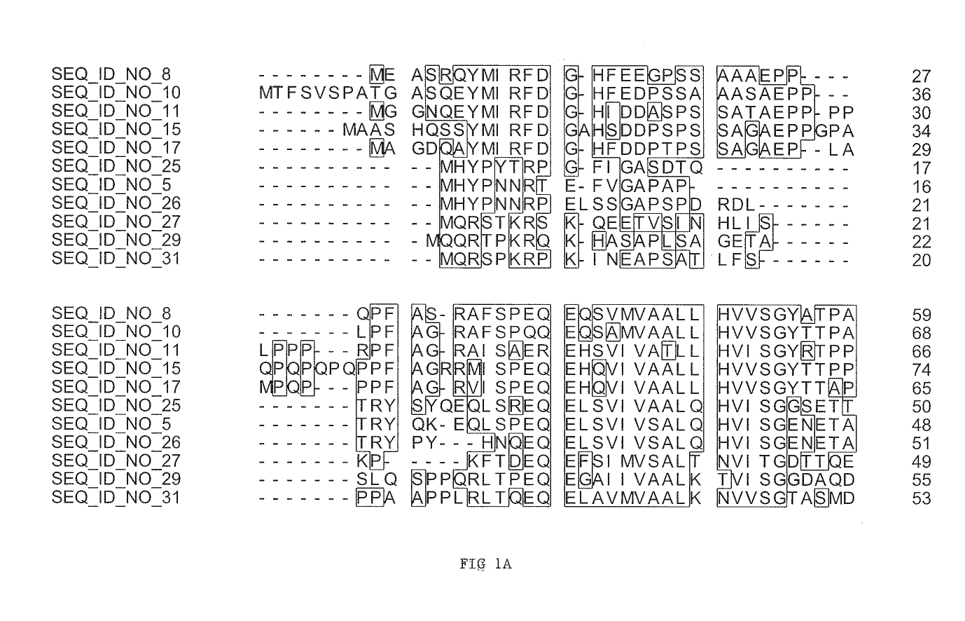

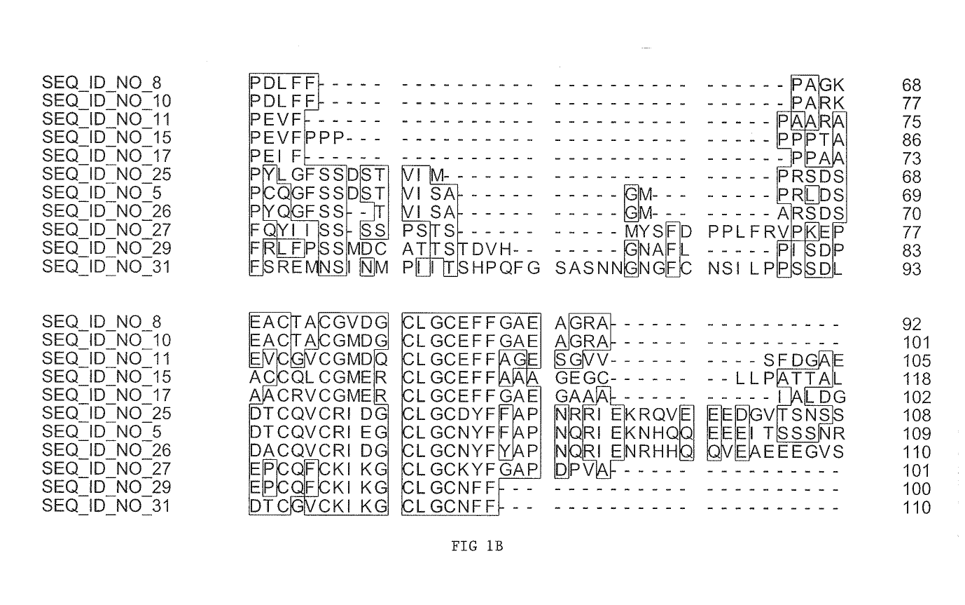

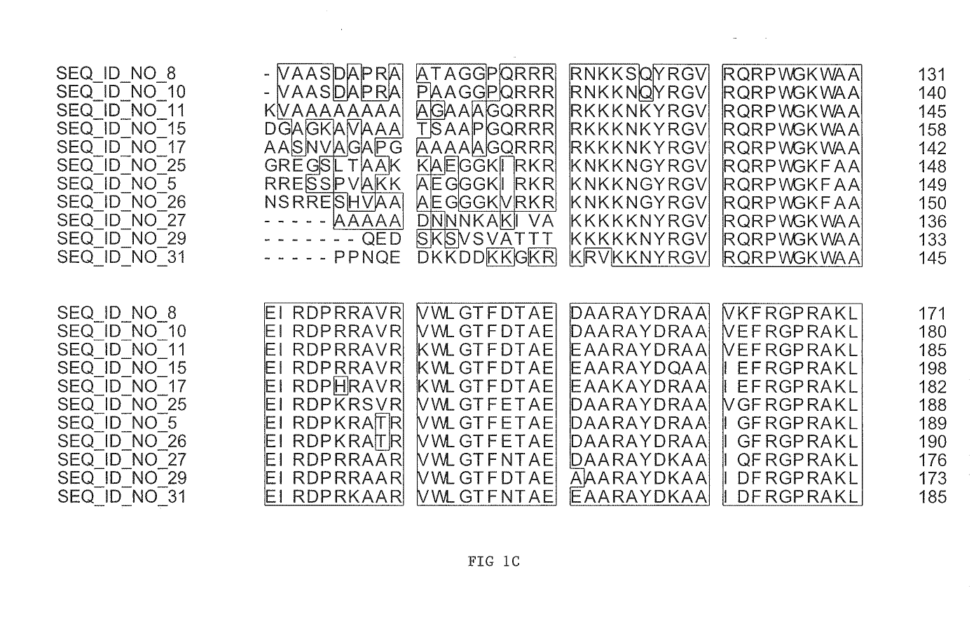

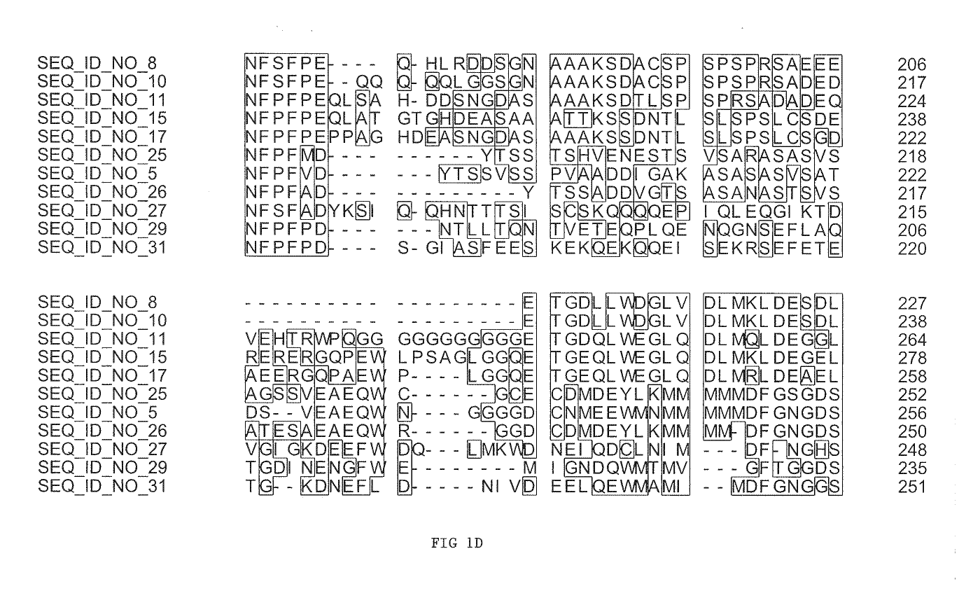

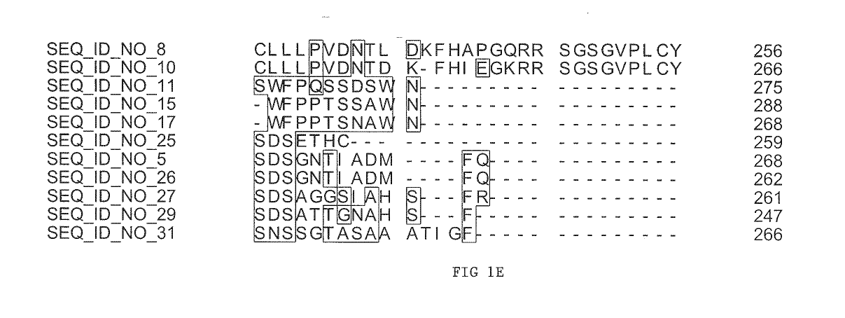

Examples of amino acid sequences of functional homologs of the polypeptide set forth in SEQ ID NO:5 are provided in FIG. 1 and in the Sequence Listing. Such functional homologs include, for example, GI ID No. 47852612 (SEQ ID NO:6), Ceres Clone ID No. 1494990 (SEQ ID NO:8), Ceres Clone ID No. 634402 (SEQ ID NO:10), GI ID No. 125603736 (SEQ ID NO:11), Ceres Annot ID No. 6318302 (SEQ ID NO:13), Ceres Annot ID No. 6014857 (SEQ ID NO:15), Ceres Clone ID No. 1824070 (SEQ ID NO:17), Ceres Clone ID No. 1805402 (SEQ ID NO: 19), Ceres Annot ID No. 6041905 (SEQ ID NO:21), GI ID No. 115479555 (SEQ ID NO:22), Ceres Annot ID No. 6325681 (SEQ ID NO:24), GI ID No. 154093739 ((SEQ ID NO:25), GI ID No. 156950515 (SEQ ID NO:26), GI ID No. 129560507 (SEQ ID NO:27), GI ID No. 129560505 (SEQ ID NO:28), GI ID No. 157341002 (SEQ ID NO:29), and Ceres Annot ID No. 1460991 (SEQ ID NO: 31). In some cases, a functional homolog of SEQ ID NO:5 has an amino acid sequence with at least 30% sequence identity, e.g., 35%, 37%, 40%, 45%, 50%, 52%, 56%, 59%, 61%, 65%, 70%, 75%, 80%, 85%, 90%, 95%, 97%, 98%, or 99% sequence identity, to the amino acid sequence set forth in SEQ ID NO:5.

In some embodiments, a plant sterility polypeptide can encode a polypeptide having a DUF640 domain. See, for example, the polypeptides set forth in SEQ ID NOs: 925, 926, 928, 930, 932, 934, 936, 938, 940, 942, 944, 946, 948, 950, 952, 954, 955, 957, 958, 959, 960, 961, 962, 963, 964, 965, 966, and 967 of U.S. Patent Application No. 61/252,827, filed Oct. 19, 2009. For example, a useful plant sterility polypeptide can have the amino acid sequence set forth in SEQ ID NO:925 of U.S. Patent Application No. 61/252,827.

The identification of conserved regions in a plant sterility polypeptide facilitates production of variants of plant sterility polypeptides. Variants of plant sterility polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. A useful variant polypeptide can be constructed based on the alignment set forth in FIG. 1 and/or homologs identified in the Sequence Listing. Such a polypeptide includes the conserved regions, arranged in the order depicted in the FIGURE from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at a position marked by dashes, such a polypeptide has a length that is the sum of the amino acid residues in all conserved regions and all dashes.

In some embodiments, useful plant sterility polypeptides include those that fit a Hidden Markov Model based on the polypeptides set forth in FIG. 1. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, —consistency REPS of 2; -ir, —iterative-refinement REPS of 100; -pre, —pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer.wustl.edu; and fr.com/hmmer232/. Hmmbuild outputs the model as a text file.

The HMM for a group of functional homologs can be used to determine the likelihood that a candidate plant sterility polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

The polypeptides discussed herein fit the indicated HMM with an HMM bit score greater than 175 (e.g., greater than 200, 300, 400, or 500). In some embodiments, the HMM bit score of a polypeptide is about 50%, 60%, 70%, 80%, 90%, or 95% of the HMM bit score of a functional homolog provided in the Sequence Listing of this application. In some embodiments, a polypeptide discussed herein fits the indicated HMM with an HMM bit score greater than 175, and has an AP2 domain, a CMX-1 motif, and a CMX-2 motif. In some embodiments, a polypeptide fits the indicated HMM with an HMM bit score greater than 175, and has 70% or greater sequence identity (e.g., 75%, 80%, 85%, 90%, 95%, or 100% sequence identity) to an AP2 domain of SEQ ID NOs: 5, 6, 8, 10, 11, 13, 15, 17, 19, 21, 22, 24, 25, 26, 27, 28, 29, or 31.

Examples of polypeptides are shown in the sequence listing that have HMM bit scores greater than 175 when fitted to an HMM generated from the amino acid sequences set forth in FIG. 1. Such polypeptides include, for example, the polypeptides set forth in SEQ ID NOs:5, 6, 8, 10, 11, 13, 15, 17, 19, 21, 22, 24, 25, 26, 27, 28, 29, and 31.

Nucleic acids encoding plant sterility polypeptides are set forth in the sequence listing. Examples of such nucleic acids include SEQ ID NOs: 4, 7, 9, 12, 14, 16, 18, 20, 23, and 30. A nucleic acid also can be a fragment that is at least 40% (e.g., at least 45, 50, 55, 60, 65, 70, 75, 80, 85, 90, 95, or 99%) of the length of the full-length nucleic acid set forth in SEQ ID NOs: 4, 7, 9, 12, 14, 16, 18, 20, 23, and 30. A nucleic acid encoding a sterility polypeptide can comprise the nucleotide sequence set forth in SEQ ID NOs: 4, 7, 9, 12, 14, 16, 18, 20, 23, and 30. Alternatively, a plant sterility nucleic acid can be a variant of the nucleic acid having the nucleotide sequence set forth in SEQ ID NOs: 4, 7, 9, 12, 14, 16, 18, 20, 23, and 30. For example, a plant sterility nucleic acid can have a nucleotide sequence with at least 80% sequence identity, e.g., 81%, 85%, 90%, 95%, 97%, 98%, or 99% sequence identity, to the nucleotide sequence set forth in SEQ ID NOs: 4, 7, 9, 12, 14, 16, 18, 20, 23, and 30.

In some embodiments, a plant sterility sequence can encode a cytotoxic polypeptide that is produced during a particular developmental stage such that establishment of spikelet meristem identity, establishment of floral meristem identity, or floral organ initiation, development, or function is affected. Non-limiting examples of cytotoxic polypeptides include a barnase polypeptide, a pectate lyase polypeptide, or a diphtheria toxin A chain polypeptide. Other cytotoxic polypeptides include small cationic molecules such as those found in venoms or skin secretions. See, e.g., Kourie and Shorthouse, Am J Physiol Cell Physiol, 278(6):C1063-C1087 (2000).

Inhibiting Expression of a Sequence of Interest

A number of nucleic acid based methods, including antisense RNA, ribozyme directed RNA cleavage, post-transcriptional gene silencing (PTGS), e.g., RNA interference (RNAi), and transcriptional gene silencing (TGS) can be used to inhibit gene expression and confer sterility in plants. Suitable polynucleotides include full-length nucleic acids encoding regulatory proteins or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid segment from a gene to be repressed is cloned and operably linked to a regulatory region and a transcription termination sequence so that the antisense strand of RNA is transcribed. The recombinant vector is then transformed into plants, as described below, and the antisense strand of RNA is produced. The nucleic acid segment need not be the entire sequence of the gene to be repressed, but typically will be substantially complementary to at least a portion of the sense strand of the gene to be repressed.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophile, can be useful. See, for example, U.S. Pat. Nos. 4,987,071 and 6,423,885.

PTGS, e.g., RNAi, can also be used to inhibit the expression of a gene. For example, the nucleotide sequences set forth in one or more of SEQ ID NOs: 1, 2, 3, and 32-39 can be used to produce RNAi constructs to inhibit gene expression. SEQ ID NOs: 1-2 are the nucleotide sequences of switchgrass homologs of AP2 domain transcription factors and ubiquitin ligase family, respectively. SEQ ID NO: 3 and 32 are chimeras containing fragments from three switchgrass homologs of MADS box domain transcription factors. Plant sterility sequences comprising all or a portion of the nucleotide sequences set forth in SEQ ID NOs: 1, 2, 3, or 32 and that are transcribed into a transcription product can be used to inhibit expression and confer sterility in switchgrass. For example, a plant sterility sequence comprising all or a portion of the nucleotide sequence set forth in SEQ ID NO: 1 affects floral organ initiation, development, or function. A plant sterility sequence comprising all or a portion of the nucleotide sequence set forth in SEQ ID NO: 3 affects floral meristem identity, or floral organ initiation, development, or function and can be used to inhibit expression and confer sterility in switchgrass. See Example 3. It will be appreciated that other portions of MADS box domain transcription factors can be used to inhibit expression and confer sterility in switchgrass.

In some embodiments, a plant sterility sequence can be transcribed into a transcription product that inhibits expression of a polypeptide containing an AP2 domain, such as AP2, IDS1 (Indeterminate Spikelet 1), SNB (Supernumerary bract, two AP2 domains), or IFA1 (indeterminate floral apex1). See, Chuck et al., Genes Dev., 12(8):1145-1154 (1998); Lee et al., Plant J., 49(1):64-78 (2006); and Laudencia-Chingcuanco and Hake, Development, 129(11):2629-38 (2002). IDS1, SNB, and IFA1 affect spikelet meristem identity while AP2 affects floral organ initiation, development, and function. SEQ ID NO:33 sets forth the nucleotide sequence of a Panicum virgatum clone, identified herein as Ceres Clone Id No. 1807588 that is predicted to encode an IDS1 polypeptide containing an AP2 domain. SEQ ID NO:35 sets forth the nucleotide sequence of a Panicum virgatum clone, identified herein as Ceres Clone Id No. 2009001 that is predicted to encode a SNB polypeptide containing two AP2 domains. SEQ ID NO:38 sets forth the nucleotide sequence of a Panicum virgatum clone, identified herein as Ceres Clone Id No. 1789568 that is predicted to encode an AP2 polypeptide.

In some embodiments, a plant sterility sequence can be transcribed into a transcription product that inhibits expression of a polypeptide having a MADS box domain, e.g., LHS1 (Leafy hull sterile 1), FUL (fruitful), PAP2 (panicle phytomer 2), AP1 (Apetela1), or CAL (Cauliflower, also known as AP1 or OsMADS14); a B-class MADS box protein such as PI (Pistillata); or a C-class MADS box protein such as AG (AGAMOUS), OsMADS58 (homolog of AG), or SPW1 (Superwoman). See, e.g., Kobayashi et al., Plant Cell Physiol., 51(1): 47-57 (2010); Jeon et al., Plant Cell., 12(6):871-84 (2000); Alvarez-Buylla et al., J Exp Bot., 57(12):3099-107 (2006); Gu et al., Development, 125(8):1509-17 (1998); Yamaguchi et al., Plant Cell, 18(1):15-28. (2006); Ohmori et al., Plant Cell, 21(10):3008-25 (2009), and Piwarzyk et al., Plant Physiol., 145(4):1495-505 (2007). PAP2 and LHS1 affect spikelet meristem identity. FUL, CAL, and AP1 affect floral meristem identity. CAL, AP1, PI, AG, OsMADS58, and SPW1 affect floral organ initiation, development, or function. The MADS box domain is found in transcription factor proteins and can bind DNA. Proteins belonging to the MADS family function as dimers, each subunit of which contributes an amphipathic alpha helix to form the anti-parallel coiled-coil DNA-binding element. The MADS-box domain is commonly associated with a K-box region, which is predicted to have a coiled-coil structure and play a role in multimer formation. SEQ ID NO:34 sets forth the nucleotide sequence of a Panicum virgatum clone, identified herein as Ceres Clone Id No. 1821199 that is predicted to encode a PAP2 polypeptide containing a MADS box domain. SEQ ID NO:36 sets forth the nucleotide sequence of a Panicum virgatum clone, identified herein as Ceres Clone Id No. 1822499 that is predicted to encode a LHS1 polypeptide containing a MADS box domain. SEQ ID NO:37 sets forth the nucleotide sequence of a Panicum virgatum clone, identified herein as Ceres Clone Id No. 1815457 that is predicted to encode an AP1 polypeptide containing a MADS box domain. SEQ ID NO:39 sets forth the nucleotide sequence of a Panicum virgatum clone, identified herein as Ceres Clone Id No. 100174842 that is predicted to encode a MADS58 polypeptide containing a MADS box domain.

In some embodiments, a plant sterility sequence can be transcribed into a transcription product that inhibits expression of a polypeptide having an F box domain, such as APO1 (aberrant panicle organization 1, SEQ ID NO:2). See, e.g., Ikeda et al., Plant J., 51(6):1030-1040 (2007). APO1 affect spikelet meristem identity. An F box domain typically is about 50 amino acids long, and is usually found in the N-terminal half of a protein. An F-box domain can include leucine rich repeats and the WD repeat. The F-box domain helps mediate protein-protein interactions in a variety of contexts, including polyubiquitination, transcription elongation, centromere binding and translational repression.

In some embodiments, a plant sterility sequence can be transcribed into a transcription product that inhibits expression of a polypeptide having an ERF (ethylene-responsive element-binding factor) domain, such as branched silkless 1 and FZP (Frizzle panicle, homolog of BD1). See, e.g., Komatsu et al., supra (2003). BD1 and FZP affect floral meristem identity. An ERF domain is found in transcription factors and can specifically bind to the GCC box AGCCGCC, which is involved in the ethylene-responsive transcription of genes. See, e.g., Komatsu et al., Development, 130:3841-3850 (2003).

In some embodiments, a plant sterility sequence can be transcribed into a transcription product that inhibits expression of a polypeptide having an N-terminal proline rich domain and a conserved C-terminal domain, such as LFY (Leafy). See, e.g., Rao et al., Proc. Natl. Acad. Sci., 105(9):3646-3651 (2008). LFY affects establishment of spikelet meristem identity and floral meristem identity.

For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence of the polypeptide of interest, or a fragment thereof, and that is from about 10 nucleotides to about 2,500 nucleotides in length. For example, the length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand, or a fragment thereof, of the coding sequence of the polypeptide of interest, and can have a length that is shorter, the same as, or longer than the corresponding length of the sense sequence. In some cases, one strand of the stem portion of a double stranded RNA comprises a sequence that is similar or identical to the 3′ or 5′ untranslated region, or a fragment thereof, of the mRNA encoding the polypeptide of interest, and the other strand of the stem portion of the double stranded RNA comprises a sequence that is similar or identical to the sequence that is complementary to the 3′ or 5′ untranslated region, respectively, of the mRNA encoding the polypeptide of interest. In other embodiments, one strand of the stem portion of a double stranded RNA comprises a sequence that is similar or identical to the sequence of an intron, or a fragment thereof, in the pre-mRNA encoding the polypeptide of interest, and the other strand of the stem portion comprises a sequence that is similar or identical to the sequence that is complementary to the sequence of the intron, or a fragment thereof, in the pre-mRNA.

The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron, or a fragment thereof. A double stranded RNA can have zero, one, two, three, four, five, six, seven, eight, nine, ten, or more stem-loop structures.

A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing a regulatory region operably linked to a nucleic acid in sense orientation can also be used to inhibit the expression of a gene. The transcription product can be similar or identical to the sense coding sequence, or a fragment thereof, of a polypeptide of interest. The transcription product can also be unpolyadenylated, lack a 5′ cap structure, or contain an unsplicable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the full-length sequence, or a fragment thereof, of an mRNA, the 3′ or 5′ untranslated region of an mRNA, or an intron in a pre-mRNA encoding a polypeptide of interest. In some embodiments, the sense or antisense sequence is identical or complementary to a sequence of the regulatory region, or a fragment thereof, that drives transcription of the gene encoding a polypeptide of interest. In each case, the sense sequence is the sequence that is complementary to the antisense sequence.

The sense and antisense sequences can be any length greater than about 12 nucleotides (e.g., 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence encoding a polypeptide described herein. The sense sequence complementary to the antisense sequence can be a sequence present within the mRNA of a polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or P-DNA such that the left and right T-DNA border sequences, or the left and right border-like sequences of the P-DNA, flank or are on either side of the nucleic acid. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some embodiments, a nucleic acid as described above is designed to inhibit expression of more than one gene in a plant. Such a nucleic acid has fragment(s) from a first gene to be inhibited as well as fragment(s) from a second, third or even fourth gene to be inhibited. For example, the nucleotide sequences set forth in SEQ ID NO: 3 and SEQ ID NO:32, which contain nucleotide sequences from three switchgrass homologs of transcription factors containing a MADS box domain, can be utilized to design nucleic acids that inhibit expression of multiple genes. In another embodiment, a construct can be used to target Shatterproof 1 (SHPT), SHP2, aintegumenta (ANT) and crabs claw (CRC). See, for example, Colombo et al., Dev Biol. 337(2):294-302 (2010), Epub 2009 Nov. 6.

Transcription factors. F₁ transgenic switchgrass plants described herein contain an exogenous nucleic acid encoding a transcription factor that activates transcription of the plant sterility sequence(s) present in such plants. A single transcription factor can activate both plant sterility sequences, each of which is operably linked to the same upstream activation sequence (UAS). Alternatively, two different transcription factors can be expressed such that each of the transcription factors activates one of the plant sterility sequences. Each sterility sequence can have a different expression pattern. For example, each transcription factor can be linked to a different promoter such that each sterility sequence can be expressed at a different developmental stage such that establishment of spikelet meristem identity, establishment of floral meristem identity, or floral organ initiation, development, or function can be affected. In some embodiments, the first transcription factor can be operably linked to a constitutive promoter and the second transcription factor can be operably linked to a vegetative promoter. In other embodiments, both transcription factors are operably linked to different vegetative promoters.

Transcription factors typically have discrete DNA binding and transcription activation domains. The DNA binding domain(s) and transcription activation domain(s) of transcription factors can be synthetic or can be derived from different sources (i.e., be chimeric transcription factors). It is known that domains from different naturally occurring transcription factors can be combined in a single polypeptide and that expression of such a chimeric transcription factor in plants can activate transcription. In some embodiments, a chimeric transcription factor has a DNA binding domain derived from the yeast Gal4 gene and a transcription activation domain derived from the VP16 gene of herpes simplex virus. In other embodiments, a chimeric transcription factor has a DNA binding domain derived from a yeast HAP1 gene and the transcription activation domain derived from VP16. See, e.g., WO 97/30164.

A list of DNA binding domains from various transcription factors is shown in Table 1, along with their respective upstream activation sequences. These domains are suitable for use in a chimeric transcription factor in switchgrass. DNA-binding domains on this list have been expressed in transgenic plants as components of chimeric transcription factors. It is contemplated that the DNA binding domain from a S. cerevisiae LEU3 transcription factor and its associated UAS (CCG-N4-CGG) and the DNA binding domain from a S. cerevisiae PDR3 transcription factor and its associated UAS (CCGCGG) will also be suitable. See, Hellauer et al. Mol. Cell. Biol. (1996).

TABLE 1
Binding Domains

| Transcription | Source | | |
|---|---|---|---|
| Factor Name | Organism | UAS | Reference |
| HAP1 | S. cerevisiae | agcaCGGacttatCGGtcgg | WO 97/30164 |
| | | (SEQ ID NO: 50) | |
| | | GcagCGGtattaaCGGgattac | |
| | | (SEQ ID NO: 51) | |
| LexA | E. coli | TACTG(TA)5CAGTA | US Pat. No. 6,399,857; |
| | | (SEQ ID NO: 52) | US Pat. No. 6,946,586; |
| | | | Wade et al, Genes & |
| | | | Dev. 19:2619-2630, 2005 |
| Lac Operon | E. coli | AATTGTGAGCGCTCACAATT | Moore et al. PNAS Jan 6; |
| | | (SEQ ID NO: 53) | 95(1): 376-81 (1998); |
| | | | US Pat. No. 6,172,279 |
| ArgR | E. coli | wNTGAAT-w4-ATTCANw | Werner K Maas, Microbiol |
| | | (SEQ ID NO: 54) | Review, 1994 Vol 58, pp. |
| | | | 631-640 |
| AraC | E. coli | TATGGATAAAAATGCTA | Bustos and Schleif, 1993 |
| | | (SEQ ID NO: 55) | |
| Synthetic Zn | N/A | N/A | US Pat. No. 7,273,923; |
| proteins | | | US Pat. No. 7,262,054 |

A list of transcription activation domains from various transcription factors is shown in Table 2, along with the amino acid residues where the domain is located in the protein. These domains are suitable for use in a chimeric transcription factor in switchgrass. Most of the activation domains on this list have been shown to be functional in heterologous plant systems.

TABLE 2
Activation Domains

| | | Domain | |
|---|---|---|---|
| | | Location | |
| Transcription | | (Amino Acid | |
| Factor Name | Organism | Residue Nos.) | Reference |
| C1 protein | Maize | 173-273 | Goff SA et al., Gene & Dev. (1991), Van |
| | | | Eenenaam et al. Metab Eng. (2004) |
| ATMYB2 | Arabidopsis | 146-269 | Urao et al., Plant J. (1996) |
| HAFL-1 | Wheat | 214-273 | Okanami et al. Genes to Cells (1996) |
| ANT | Arabidopsis | 221-274 | Krizek & Sulli, Planta (2006) |
| ALM2 | Arabidopsis | 203-256 | Anderson & Hanson, BMC Plant Biol. |
| | | | (2005) |
| AvrXa10 | Xanthomonas | 133-274 | Zhu et al. Plant Cell 1999 |
| | oryzae pv. | | |
| | oryzae | | |
| Viviparous 1 | Maize | 134-213 | McCarty et al. Cell (1991) |
| (VP1) | | | |
| DOF | Maize | 1-163 | Yanagisawaa & Sheen Plant Cell (1998) |
| RISBZ1 | Rice | 1060-1102 | Onodera et al., J. Biol. Chem. (2001) |
| VP16 | Herpes simplex | 411-490 | Greaves and O'Hare, J. Virol., 63:1641- |
| | | | 1650 (1989) |

Regulatory Regions

The choice of regulatory regions to be included in a recombinant construct depends upon several factors, including, but not limited to, efficiency, selectability, inducibility, desired expression level, and cell- or tissue-preferential expression. For example, to affect the establishment of spikelet meristem identity, a promoter such as PD3796 (SEQ ID NO:40) or PD3800 (SEQ ID NO:41), or functional fragments thereof, can be used in a nucleic acid construct. To affect the establishment of floral meristem identity, a promoter such as CeresAnnot:8643934 (SEQ ID NO:42), CeresAnnot:8632648 (SEQ ID NO:43), CeresAnnot:8681303 (SEQ ID NO:44), or CeresAnnot:8642422 (SEQ ID NO:45), or functional fragments thereof, can be used in a nucleic acid construct. To affect floral organ initiation, development, or function, a promoter such as CeresAnnot:8657974 (SEQ ID NO:46), CeresAnnot:8732691 (SEQ ID NO:47), CeresAnnot:8031970 (SEQ ID NO:48), or CeresAnnot:8669907 (SEQ ID NO:49), or functional fragments thereof, can be used in a nucleic acid construct. It is a routine matter for one of skill in the art to position regulatory regions relative to the coding sequence and to identify functional fragments of regulatory regions.

For example, methods for identifying and characterizing regulatory regions in plant genomic DNA, include those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996). In one embodiment, the ability of regulatory regions of varying lengths to direct expression of an operably linked nucleic acid can be assayed by operably linking varying lengths of a regulatory region to a reporter nucleic acid and transiently or stably transforming a cell, e.g., a plant cell, with such a construct. Suitable reporter nucleic acids include β-glucuronidase (GUS), green fluorescent protein (GFP), yellow fluorescent protein (YFP), and luciferase (LUC). Expression of the gene product encoded by the reporter nucleic acid can be monitored in such transformed cells using standard techniques.

Examples of various classes of regulatory regions are described below. Some of the regulatory regions indicated below as well as additional regulatory regions are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; and PCT/US07/62762.

For example, the sequences of regulatory regions p326, PD2995, PD3141, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PR0924 is set forth in the sequence listing of PCT/US07/62762; the sequences of regulatory regions p530c10, pOsFIE2-2, pOsMEA, pOsYp102, and pOsYp285 are set forth in the sequence listing of PCT/US06/038236; the sequence of PD2995 is set forth in the sequence listing of PCT/US09/32485; and the sequence of PD3141 promoter is set forth in the sequence listing of PCT/US09/32485.

It will be appreciated that a regulatory region may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326, PD2995, YP0144, YP0190, p13879, YP0050, p32449, 21876, YP0158, YP0214, YP0380, PT0848, and PT0633 promoters. Additional examples include the cauliflower mosaic virus (CaMV) 35S promoter, the mannopine synthase (MAS) promoter, the l′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380 and PT0585.

Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380, PT0848, YP0381, YP0337, PT0633, YP0374, PT0710, YP0356, YP0385, YP0396, YP0388, YP0384, PT0688, YP0286, YP0377, PD1367, and PD0901. Examples of nitrogen-inducible promoters include PT0863, PT0829, PT0665, and PT0886. Examples of shade-inducible promoters include PR0924 and PT0678. An example of a promoter induced by salt is rd29A (Kasuga et al. (1999) Nature Biotech 17: 287-291).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, shoot-preferential, parenchyma cell-preferential, and senescence-preferential promoters. In some embodiments, a promoter may preferentially drive expression in reproductive tissues (e.g., PO2916 promoter, SEQ ID NO:31 in 61/364,903). Promoters designated YP0086, YP0188, YP0263, PT0758, PT0743, PT0829, YP0119, and YP0096, as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a heat and/or drought-tolerance polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Nucleic acid expression. For expression of a plant sterility sequence, a suitable nucleic acid encoding a gene product is operably linked to a promoter and a UAS for a transcription factor. For expression of a transcription factor, a transcription factor coding sequence is operably linked to a promoter. As used herein, the term “operably linked” refers to positioning of a regulatory region in a nucleic acid so as to allow or facilitate transcription of the nucleic acid to which it is linked. For example, a recognition site for a transcription factor is positioned with respect to a promoter so that upon binding of the transcription factor to the recognition site, the level of transcription from the promoter is increased. The position of the recognition site relative to the promoter can be varied for different transcription factors, in order to achieve the desired increase in the level of transcription. Selection and positioning of promoter and transcription factor recognition site is affected by several factors, including, but not limited to, desired expression level, cell or tissue specificity, and inducibility.

A nucleic acid for use in the invention may be obtained by, for example, DNA synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach, C. & Dveksler, G., Eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are available by which site-specific nucleotide sequence modifications can be introduced into a template nucleic acid.

Nucleic acids for use in the invention may be detected by techniques such as ethidium bromide staining of agarose gels, Southern or Northern blot hybridization, PCR or in situ hybridizations. Hybridization typically involves Southern or Northern blotting. See e.g., Sambrook et al., 1989, Molecular Cloning: A Laboratory Manual, 2^{nd} Edition, Cold Spring Harbor Press, Plainview, N.Y., sections 9.37-9.52. Probes should hybridize under high stringency conditions to a nucleic acid or the complement thereof. High stringency conditions can include the use of low ionic strength and high temperature washes, for example 0.015 M NaCl/0.0015 M sodium citrate (0.1×SSC), 0.1% sodium dodecyl sulfate (SDS) at 65° C. In addition, denaturing agents, such as formamide, can be employed during high stringency hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Herbicide Tolerance

In addition to the other exogenous nucleic acids described herein, switchgrass plants typically contain a transgene that confers herbicide resistance. Herbicide resistance is also sometimes referred herein to as herbicide tolerance. Expression of a herbicide resistance transgene is regulated independently of plant sterility sequences in plants, i.e., is not regulated by transcription factors encoded by exogenous nucleic acids. Polypeptides conferring resistance to a herbicide that inhibits the growing point or meristem, such as an imidazolinone or a sulfonylurea can be suitable. Exemplary polypeptides in this category code for mutant ALS and AHAS enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS) resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides for resistance to glyphosate (sold under the trade name Roundup®) are also suitable. See, for example, U.S. Pat. No. 4,940,835 and U.S. Pat. No. 4,769,061. U.S. Pat. No. 5,554,798 discloses transgenic glyphosate resistant maize plants, in which resistance is conferred by an altered 5-enolpyruvyl-3-phosphoshikimate (EPSP) synthase. Such polypeptides can confer resistance to glyphosate herbicidal compositions, including without limitation glyphosate salts such as the trimethylsulphonium salt, the isopropylamine salt, the sodium salt, the potassium salt and the ammonium salt. See, e.g., U.S. Pat. Nos. 6,451,735 and 6,451,732.

Polypeptides for resistance to phosphono compounds such as glufosinate ammonium or phosphinothricin, and pyridinoxy or phenoxy propionic acids and cyclohexones are also suitable. See European application No. 0 242 246. See also, U.S. Pat. Nos. 5,879,903, 5,276,268 and 5,561,236.

Other herbicides include those that inhibit photosynthesis, such as a triazine and a benzonitrile (nitrilase). See U.S. Pat. No. 4,810,648. Other herbicides include 2,2-dichloropropionic acid, sethoxydim, haloxyfop, imidazolinone herbicides, sulfonylurea herbicides, triazolopyrimidine herbicides, s-triazine herbicides and bromoxynil. Also suitable are herbicides such as isoxazoles that inhibit hydroxyphenylpyruvate dioxygenases. Also suitable are herbicides that confer resistance to a protox enzyme. See, e.g., U.S. Patent Application No. 20010016956, and U.S. Pat. No. 6,084,155.

Transformation

Techniques for introducing exogenous nucleic acids into switchgrass plants include, without limitation, Agrobacterium-mediated transformation and particle gun transformation. See, e.g., Richards et al., Plant Cell. Rep. 20:48-54 (2001) and Somleva et al., Crop Sci. 42:2080-2087 (2002). If a cell or tissue culture is used as the recipient tissue for transformation, plants can be regenerated from transformed cultures by techniques known to those skilled in the art.

### IV. SEQUENCES OF INTEREST

Switchgrass cells and plants described herein can also have an exogenous nucleic acid that comprises a sequence of interest, which is preselected for its beneficial effect upon a trait of commercial value. An exogenous nucleic acid comprising a sequence of interest is operably linked to a regulatory region for transformation into switchgrass plants, and plants are selected whose expression of the sequence of interest achieves a desired amount and/or specificity of expression. A suitable regulatory region is chosen as described herein. In most cases, expression of a sequence of interest is regulated independently of plant sterility sequences in plants, i.e., is not regulated by exogenous nucleic acids encoding transcription factors as described herein. It will be appreciated, however, that in some embodiments expression of a sequence of interest is regulated by transcription factors that regulate plant sterility sequences as described herein.

A sequence of interest can encode a polypeptide or can regulate the expression of a polypeptide. A sequence of interest that encodes a polypeptide can encode a plant polypeptide, a non-plant polypeptide such as a mammalian polypeptide, a modified polypeptide, a synthetic polypeptide, or a portion of a polypeptide. In some embodiments, a sequence of interest is transcribed into an antisense or interfering RNA molecule.

More than one sequence of interest can be present in a plant, e.g., two, three, four, five, six, seven, eight, nine, or ten sequences of interest can be present in a plant. Each sequence of interest can be present on the same nucleic acid construct or can be present on separate nucleic acid constructs. The regulatory region operably linked to each sequence of interest can be the same or can be different.

Lignin Biosynthesis Sequences

In certain cases, a sequence of interest can be an endogenous or exogenous sequence associated with lignin biosynthesis. For example, transgenic switchgrass containing a recombinant nucleic acid encoding a regulatory protein can be effective for modulating the amount and/or rate of lignin biosynthesis. Such effects on lignin biosynthesis typically occur via modulation of transcription of one or more endogenous or exogenous sequences of interest operably linked to an associated regulatory region, e.g., endogenous genes involved in lignin biosynthesis, such as native enzymes or regulatory proteins in lignin biosynthesis pathways, or exogenous sequences involved in lignin biosynthesis pathways introduced via a recombinant nucleic acid construct into a plant cell.

In some embodiments, the coding sequence can encode a polypeptide involved in lignin biosynthesis, e.g., an enzyme or a regulatory protein (such as a transcription factor) involved in lignin biosynthesis described herein. Other components that may be present in a sequence of interest include introns, enhancers, upstream activation regions, and inducible elements.

A suitable sequence of interest can encode an enzyme involved in lignin biosynthesis, such as 4-(hydroxy)cinnamoyl CoA ligase (4CL; EC 6.2.1.12), p-coumarate 3-hydroxylase (C3H), cinnamate 4-hydroxylase (C4H; EC 1.14.13.11), cinnamyl alcohol dehydrogenase (CAD; EC 1.1.1.195), caffeoyl CoA O-methyltransferase (CCoAOMT; EC 2.1.1.104), cinnamoyl CoA reductase (CCR; EC 1.2.1.44), caffeic acid/5-hydroxyferulic acid O-methyltransferase (COMT; EC 2.1.1.68), hydroxycinnamoyl CoA:quinate hydroxycinnamoyltransferase (CQT; EC 2.3.1.99), hydroxycinnamoyl CoA:shikimate hydroxycinnamoyltransferase (CST; EC 2.3.1.133), ferulate 5-hydroxylase (F5H), phenylalanine ammonia-lyase (PAL; EC 4.3.1.5), p-coumaryl CoA 3-hydroxylase (pCCoA3H), or sinapyl alcohol dehydrogenase (SAD).

In some embodiments, a suitable sequence of interest can encode an enzyme involved in polymerization of lignin monomers to form lignin, such as a peroxidase (EC 1.11.1.x) or a laccase (EC 1.10.3.2) enzyme. In some cases, a suitable sequence of interest can encode an enzyme involved in glycosylation of lignin monomers, such as a coniferyl-alcohol glucosyltransferase (EC 2.4.1.111) enzyme, or an enzyme involved in regenerating a monolignol from a monolignol glucoside, such as a coniferin β-glucosidase (EC 3.2.1.126) enzyme. As mentioned above, such a suitable sequence of interest can be transcribed into an anti-sense or interfering RNA molecule.

Phenylpropanoid Sequences of Interest

In some embodiments, a sequence of interest can encode an enzyme involved in flavonoid biosynthesis, such as naringenin-chalcone synthase (EC 2.3.1.74), polyketide reductase, chalcone isomerase (EC 5.5.1.6), flavanone 4-reductase (EC 1.1.1.234), dihydrokaempferol 4-reductase (EC 1.1.1.219), flavone synthase (EC 1.14.11.22), flavone 7-O-beta-glucosyltransferase (EC 2.4.1.81), flavone apiosyltransferase (EC 2.4.2.25), isoflavone-7-O-beta-glucoside 6″-O-malonyltransferase (EC 2.3.1.115), apigenin 4′-O-methyltransferase (EC 2.1.1.75), flavonoid 3′-monooxygenase (EC 1.14.13.21), luteolin O-methyltransferase (EC 2.1.1.42), flavonoid 3′,5′-hydroxylase (EC 1.14.13.88), 4′-methoxyisoflavone 2′-hydroxylase (EC 1.14.13.53), isoflavone 4′-O-methyltransferase (EC 2.1.1.46), flavanone 3-dioxygenase (EC 1.14.11.9), leucocyanidin oxygenase (EC 1.14.11.19), flavonol synthase (EC 1.14.11.23), 2′-hydroxyisoflavone reductase (EC 1.3.1.45), leucoanthocyanidin reductase (EC 1.17.1.3), anthocyanidin reductase (EC 1.3.1.77), flavonol 3-O-glucosyltransferase (EC 2.4.1.91), quercetin 3-O-methyltransferase (EC 2.1.1.76), anthocyanidin 3-O-glucosyltransferase (EC 2.4.1.115), flavonol-3-O-glucoside L-rhamnosyltransferase (EC 2.4.1.159), UDP-glucose:anthocyanin 5-O-glucosyltransferase (2.4.1.-), or anthocyanin acyltransferase (2.3.1.-).

In some embodiments, a sequence of interest can encode an enzyme involved in stilbene synthesis such as trihydroxystilbene synthase (EC 2.3.1.95) or an oxidoreductase (EC 1.14.-.-). In some embodiments, a sequence of interest can encode an enzyme involved in coumarin synthesis such as trans-cinnamate 2-monooxygenase (EC 1.14.13.14), 2-coumarate O-beta-glucosyltransferase (EC 2.4.1.114), a cis-trans-isomerase (EC 5.2.1.-), or a beta-glucosidase (EC 3.2.1.21).

Biomass-Modulating Sequences of Interest

Sequences of interest include those encoding a biomass-modulating polypeptide that contains at least one domain indicative of biomass-modulating polypeptides.

For example, a biomass-modulating polypeptide can contain a polyprenyl synthetase domain, which is predicted to be characteristic of a polyprenyl synthetase enzyme. A polyprenyl synthetase is a variety of isoprenoid compound which can be synthesized by various organisms. For example, in eukaryotes the isoprenoid biosynthetic pathway can be responsible for the synthesis of a variety of end products including cholesterol, dolichol, ubiquinone or coenzyme Q. In bacteria, this pathway can lead to the synthesis of isopentenyl tRNA, isoprenoid quinones, and sugar carrier lipids. Among the enzymes that can participate in that pathway, are a number of polyprenyl synthetase enzymes which catalyze a 1′4-condensation between 5 carbon isoprene units. All the above enzymes typically share some regions of sequence similarity. Two of these regions are typically rich in aspartic-acid residues and could be involved in the catalytic mechanism and/or the binding of the substrates.

A biomass-modulating polypeptide can contain a multiprotein bridging factor 1 domain. This domain forms a heterodimer with MBF2. It can make direct contact with the TATA-box binding protein (TBP) and can interact with Ftz-F1, stabilising the Ftz-F1-DNA complex. It can also be found in the endothelial differentiation-related factor (EDF-1). The domain can be found in a wide range of eukaryotic proteins including metazoans, fungi and plants. A helix-turn-helix motif (PF01381) is typically found to its C-terminus.

A biomass-modulating polypeptide can contain a Helix-turn-helix 3 domain. DNA binding helix-turn helix proteins include bacterial plasmid copy control protein, bacterial methylases, various bacteriophage transcription control proteins and a vegetative specific protein from Dictyostelium discoideum (Slime mold).

A biomass-modulating polypeptide can contain a plant neutral invertase domain, such as Bac_rhamnosid, GDE_C, Invertase_neut, and Trehalase.

A biomass-modulating polypeptide can contain a sedlin, N-terminal domain. Sedlin is a 140 amino-acid protein with a role in endoplasmic reticulum-to-Golgi transport.

A biomass-modulating polypeptide can contain a G-box binding protein MFMR domain. The domain is typically found to the N-terminus of the PF00170 transcription factor domain. It is typically between 150 and 200 amino acids in length. The N-terminal half is typically rather rich in proline residues and has been termed the PRD (proline rich domain) whereas the C-terminal half is typically more polar and has been called the MFMR (multifunctional mosaic region). This family may be composed of three sub-families called A, B and C classified according to motif composition. Some of these motifs may be involved in mediating protein-protein interactions. The MFMR region can contain a nuclear localisation signal in bZIP opaque and GBF-2. The MFMR also can contain a transregulatory activity in TAF-1. The MFMR in CPRF-2 can contain cytoplasmic retention signals.

A biomass-modulating polypeptide can contain a bZIP_{—}1 transcription factor domain. The basic-leucine zipper (bZIP) transcription factors of eukaryotic cells are proteins that contain a basic region mediating sequence-specific DNA-binding followed by a leucine zipper region required for dimerization.

A biomass-modulating polypeptide can contain a bZIP_{—}2 basic region leucine zipper domain. The basic-leucine zipper (bZIP) transcription factors of eukaryotic cells are proteins that contain a basic region mediating sequence-specific DNA-binding followed by a leucine zipper region required for dimerization.

A biomass-modulating polypeptide can contain an epimerase domain. An epimerase domain is typical of a family of proteins that typically utilise NAD as a cofactor. The proteins in this family can use nucleotide-sugar substrates for a variety of chemical reactions. The proteins in this family can use nucleotide-sugar substrates for a variety of chemical reactions.

Amino acid sequences for certain biomass-modulating polypeptides discussed above and domains indicative of biomass-modulating polypeptides, are described in more detail in U.S. Application Ser. No. 61/097,789.

A biomass-modulating polypeptide can encode a D of transcription factor polypeptide. Dof transcription factors belong to a family of DNA binding proteins found in diverse plant species. Members of the D of family comprise a D of domain, which is characterized by a conserved region of about 50 amino acids with a C2-C2 finger structure associated with a basic region. See, e.g., Proc. Natl. Acad. Sci. USA 101:7833-7838 (2004).

Other Sequences of Interest

Other sequences of interest that can be used in the methods described herein include, but are not limited to, sequences encoding genes or fragments thereof that modulate cold tolerance, frost tolerance, heat tolerance, drought tolerance, water used efficiency, nitrogen use efficiency, pest resistance, biomass, chemical composition, plant architecture, and/or biofuel conversion properties. In particular, exemplary sequences are described in the following applications which are incorporated herein by reference in their entirety: US20080131581, US20080072340, US20070277269, US20070214517, US 20070192907, US 20070174936, US 20070101460, US 20070094750, US20070083953, US 20070061914, US20070039067, US20070006346, US20070006345, US20060294622, US20060195943, US20060168696, US20060150285, US20060143729, US20060134786, US20060112454, US20060057724, US20060010518, US20050229270, US20050223434, US20030217388, WO 2011/011412, WO 2010/033564, and WO2009/102965.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in switchgrass is obtained, using appropriate codon usage bias tables.

### V. SWITCHGRASS BREEDING

In some embodiments, the breeding programs described herein use genetic polymorphisms in a marker assisted breeding program to facilitate the development of parents that retain desired characteristics. One or more individual plants in a breeding program are identified that possess one or more genetic polymorphisms that are correlated with the desired characteristic. Those plants are then advanced in the breeding program. In most breeding programs, analysis for a particular polymorphic allele will be carried out in each generation, although analysis can be carried out in alternate generations if desired.

Genetic polymorphisms that are useful in such methods include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. If the probes flank an SSR in the population, PCR products of different sizes will be produced. SSR polymorphisms can also be identified by using PCR product(s) as a probe against Southern blots from different individuals in the population.

In some cases, marker-assisted selection for other useful traits is also carried out, e.g., selection for fungal resistance or bacterial resistance. Selection for such other traits can be carried out before, during or after identification of individual plants that possess the desired polymorphic allele(s).

### VI. ARTICLES OF MANUFACTURE

A plant seed composition can contain a plurality of F₁ hybrid sterile transgenic switchgrass seeds. The proportion of such seeds in the composition is from 70% to 100%, e.g., 90%, 91%, 92%, 93%, 94%, 95%, 96%, 97%, 98%, or 99% to 100%. The remaining seeds in the composition are typically seeds of one of the parents of the F₁, and the proportion of parent seeds is less than 5%, e.g., 0% to 0.5%, 1%, 2%, or 4%. The proportion of seeds in the composition is measured as the number of seeds of a particular type divided by the total number of seeds in the composition. When large quantities of a seed composition are formulated, or when the same composition is formulated repeatedly, there may be some variation in the proportion of each type observed in a sample of the composition, due to sampling error. In the present invention, such sampling error typically is about ±5%.

Typically, seeds are conditioned and bagged in packaging material by means known in the art to form an article of manufacture. Such a bag of seed preferably has a package label accompanying the bag, e.g., a tag or label secured to the packaging material, a label printed on the packaging material or a label inserted within the bag. The package label indicates that the seeds therein are F₁ hybrid sterile transgenic switchgrass seeds. The package label may indicate that plants grown from such seeds are suitable for making an indicated preselected polypeptide. The package label also may indicate the seeds contained therein incorporate transgenes that provide biological containment or confinement of plants grown from the seeds.

### VII. USES AND ADVANTAGES

Sterile switchgrass hybrids provided herein have various uses in the agricultural and energy production industries. For example, switchgrass plants described herein can be used to make animal feed and food products. Such plants, however, are often particularly useful as a feedstock for energy production.

The effect of the plant sterility sequences described herein on sterility of switchgrass hybrids can be advantageously scored by field observation, because the relative penetrance of the sterility phenotype of each plant sterility sequence can be visually scored. Consequently, the extent to which environmental and/or other factors influence sterility can be readily assessed, reducing the need for other, more time-consuming and expensive types of analyses.

Moreover, transgenic sterile switchgrass hybrids comprising plant sterility sequences as described herein beneficially permit biomass harvest at a later date in the growing season, relative to switchgrass hybrids lacking such plant sterility sequences. Harvesting biomass later in a growing season allows senescence to proceed further than would otherwise be the case, and thereby increases the amounts of nutrients transferred from above ground plant parts to the roots, which aids vegetative growth in subsequent growing seasons. In addition, senesced biomass often has a better compositional and moisture profile for a number of biofuel processing applications.

Sterile switchgrass plants described herein often produce higher yields of biomass per hectare, relative to known, non-sterile switchgrass varieties. For example, F₁ switchgrass plants grown from F₁ seeds described herein can have a statistically significant increase in biomass in the second or subsequent growing seasons relative to control F₁ switchgrass plants that lack the exogenous nucleic acids for plant sterility sequences and transcriptions factors. In some embodiments, F₁ sterile switchgrass plants provide equivalent yields of biomass per hectare relative to known switchgrass varieties when grown under conditions of reduced inputs such as fertilizer and/or water. Thus, such switchgrass plants can be used to provide yield stability at a lower input cost and/or under environmentally stressful conditions such as drought. In some embodiments, F₁ switchgrass plants described herein have a composition that permits more efficient processing into free sugars, and subsequently ethanol, for energy production. In some embodiments, such plants provide higher yields of ethanol, other biofuel molecules, and/or sugar-derived co-products per kilogram of plant material, relative to control plants.

Biomass can include harvestable plant tissues such as leaves, stems, and reproductive structures, or all plant tissues such as leaves, stems, roots, and reproductive structures. In some embodiments, biomass encompasses only above ground plant parts. In some embodiments, biomass encompasses only stem plant parts. In some embodiments, biomass encompasses only above ground plant parts except inflorescence and seed parts of a plant. Biomass can be quantified as dry matter yield, which is the mass of biomass produced (usually reported in Tons/acre) if the contribution of water is subtracted from the fresh matter weight. Dry matter yield (DMY) yield is calculated using the fresh matter weight (FMW) and a measurement of weight percent moisture (M) in the following equation: DMY=(100−M)/100)*FMW. Biomass can be quantified as fresh matter yield, which is the mass of biomass produced (usually reported in Tons/acre) on an as-received basis, which includes the weight of moisture.

The commercial production of seeds for growing switchgrass plants normally involves four stages, the production of breeder, foundation, certified and registered seeds. Breeder seed is the initial increase of seed of the variety which is developed by the breeder and from which foundation seed is derived. Foundation seed is the second generation of seed increase and from which certified seed is derived. Certified seeds are used in commercial crop production and are produced from foundation or certified seed. Foundation seed normally is distributed by growers or seedsmen as planting stock for the production of certified seed.

The sterile F₁ switchgrass hybrids described herein advantageously are produced without the need to apply any sort of chemical inducer or chemical ligand to induce sterility. The F₁ sterile hybrids exhibit an increased uniformity in phenotype relative to open-pollinated switchgrass varieties, which facilitates production operations and harvesting dates for growers.

### VIII EXAMPLES

### Example 1

### Transgenic Switchgrass Plants

The following symbols are used in with respect to transformations: T₀: plant regenerated from transformed tissue culture; T₁: first generation progeny of self-pollinated T₀ plants; T₂: second generation progeny of self-pollinated T₁ plants; T₃: third generation progeny of self-pollinated T₂ plants.

T-DNA binary vectors were introduced into switchgrass (A26 or A10 clonally propagated lines) by Agrobacterium-mediated transformation essentially as described in Richards et al., Plant Cell. Rep. 20:48-54 (2001) and Somleva et al., Crop Sci. 42:2080-2087 (2002). At least two independent events from each transformation were selected for further study; these events were referred to as switchgrass screening lines. T1 and T2 plants were grown in a field. The presence of each construct was confirmed by PCR.

Switchgrass plants were evaluated in the U.S. under greenhouse and field conditions. Under greenhouse conditions, ten plants were grown per transgenic event within one row. Visual observations were made of overall plant development and flower development. Data for plant morphology, plant height, panicle number and seed number were collected in some cases. A general estimation of plant fertility was made based on all plants of each event.

### Example 2

### Results for Ceres Clone ID 123905 (SEQ ID NO: 5)

Construct 1 contained the PO2916 promoter fused to a nucleic acid (SEQ ID NO:4) encoding 123905 (SEQ ID NO:5). The PO2916 promoter is an approximately 3 kB genomic fragment from rice located 5′ of rice gene Os02g32030, that drives expression preferentially in reproductive tissues.

Three events were produced using the PO2916:123905 transgene. All three events were strongly affected with an anthesis defect, i.e., flowers did not open. The phenotype was readily apparent from the lack of orange color, which correlates with the inability of the anthers to emerge from the flowers. Greenhouse data from these events indicate 99+% anthesis defect (i.e., an anthesis defect score of 5 as scored below). These same events in the field show 95%+anthesis defect (i.e., an anthesis defect score of 5 as scored below). Table 3 contains the plant height data collected for the three events produced with the transgene PO2916:123905.

TABLE 3
Plant Height Data for PO2916:123905 Events

| Identifier | Average Plant Height (cm)^{a} |
|---|---|
| A26 wild type^{b} | 51.5 |
| TS1-00008 | 59 |
| TS1-00009 | 62.2 |
| TS1-00010 | 57.8 |

^{a}Plant height from 18 clones were measured in the field and averaged.
^{b}transgenic lines are in the same clonal genetic background as wild type A26.

Construct 2 contained the PD2995 promoter (SEQ ID NO:21 in PCT/US09/32485) fused to a nucleic acid (SEQ ID NO:4) encoding 123905 (SEQ ID NO:5). Ten events were generated with the PD2995:123905 transgene. On a scale from 1 (wild type) to 5 (100% anthesis defect), a fairly even distribution from 1 to 5 was observed with the PD2995:123905 transgene (see Table 4) under greenhouse conditions.

TABLE 4
Data for PD2995:123905 events

| Event | Height | Tiller | Anthesis | |
|---|---|---|---|---|
| # | (cm) | # | Defect Score | Notes |
| 1 | 145 | 20 | 5 | No open flowers |
| 2 | 158 | 37 | 4 | Some open flowers |
| 3 | 168 | 40 | 2 | 25-50% of plant has open |
| | | | | flowers |
| 4 | 158 | 17 | 3 | 5-10% of plant has open |
| | | | | flowers |
| 5 | 152 | 35 | 4 | only 4 open flowers |
| 6 | 158 | 30 | 3 | 5-10% of plant has open |
| | | | | flowers |
| 7 | 155 | 30 | 3 | 5-10% of plant has open |
| | | | | flowers |
| 8 | 124 | 25 | 2 | 25-50% of plant has open |
| | | | | flowers |
| 9 | 147 | 40 | 5 | No open flowers |
| 10 | 135 | 50 | 1 | Plant is completely |
| | | | | flowering |

The results of Table 4 indicate that there is no significant negative correlation between the anthesis defect and plant height or tiller #, two measures of available biomass, for PD2995:123905. First year data on plant height may not reflect the height of a mature stand in the second and third years. However, these data suggest that the transgene may not induce a negative phenotype.

Data also were obtained from plants grown in the field. For control plants (non-transgenic A10 genetic background), there were thirty-three plots containing three plants each. From each of the ninety-nine plants, six panicles were harvested, for a total of 594 panicles. The average seed yield per panicle was 76 seed. The range of seed yield averages per plot was between 29 seed/panicle to 150 seed per panicle.

For transgenic plants produced using the PO2916:123905 transgene, panicle morphology and spikelet density were similar to controls. For each of the three PO2916:123905 events, there were three plots containing six plants each. From each of these eighteen plants, six panicles were harvested, for a total of 108 panicles. The average seed yield per panicle is shown in Table 5. There was no difference in panicle morphology between the transgenic A26 and wildtype A10 lines by visual inspection.

TABLE 5
Field Data for PO2916:123905 Events

| Identifier | Average Seed Yield/Panicle |
|---|---|
| TS1-00008 | 6.5 |
| TS1-00009 | 6.6 |
| TS1-00010 | 3.9 |

### Example 3

### Results with RNAi Constructs

Transgenic switchgrass also were produced using RNAi constructs. The FZP construct contained the PD3141 promoter (SEQ ID NO: 23 in PCT/US09/32485) and the nucleic acid sequence set forth in SEQ ID NO:1. The AG construct contained the PD3141 promoter and the nucleic acid sequence set forth in SEQ ID NO:3. The AG RNAi construct contains an amalgam of three targeting sequences that are designed to knock down the expression of three distinct members of the AG-clade of MADS-box transcription factors.

Thirty (30) events were generated with the FZP construct. Reduced fertility was observed in two of these events, with one event having visibly greater reduced fertility than the second. In the most severe representation of the phenotype, the spikelets were not produced, and the tissue that should give rise to spikelets instead gave rise to additional panicle branch material. Neither was 100% sterile. Plants from both of these events were significantly reduced in stature compared to transgenics that displayed no reduced fertility.

With the AG construct, 48 total events were generated from the transformation. From these events, two phenotypes were observed. The first phenotype was an obvious floral anthesis defect. The second phenotype was an abortion of floral organ development (i.e. anthers, ovules, and stigma were smaller than wild-type; the organs ranged from 25% to 75% of wild-type).

From the 48 events, six had significant anthesis failure (fewer than 10% of florets opened). These six events, plus an additional ten events that did not display a significant anthesis defect, were then scored for floral organ development as follows: Level 1, nearly wild-type; Level 2, <10% anthesis, at least half or more of the remaining spikelets are bulging and the floral development is equal or greater than 75% wt; Level 3, <1% anthesis, the majority of spikelets are not bulging and floral development is 25% to 75% of wild-type; Level 4, no anthesis detected at all, the majority of spikelets have 75% or more of wild-type development; Level 5, no anthesis detected at all, organ development at less than 50% wild-type development. Table 6 contains the floral organ development score and plant heights for the six plants with significant anthesis defect (<10% anthesis) as well as the 10 plants that did not display significant anthesis defects. It appears that the height of plants with reduced fertility was in the same range as that of plants with a nearly wild type phenotype. Due to the large range in plant heights, however, no significant correlations were observed between fertility level and plant height.

TABLE 6

| | | Floral organ | |
|---|---|---|---|
| | | development | Plant height |
| Seed Line ID | Event # | score | (cm) |
| PV00357 | 1 | 1 | 115 |
| PV00357 | 6 | 1 | 102 |
| PV00357 | 18 | 1 | 140 |
| PV00357 | 19 | 1 | 133 |
| PV00357 | 21 | 1 | 120 |
| PV00357 | 22 | 1 | 95 |
| PV00357 | 23 | 1 | 130 |
| PV00357 | 25 | 1 | 94 |
| PV00357 | 26 | 1 | 122 |
| PV00357 | 28 | 1 | 107 |
| PV00286 | 5 | 2 | 128 |
| PV00286 | 9 | 4 | 140 |
| PV00357 | 9 | 4 | 85 |
| PV00357 | 11 | 4 | 112 |
| PV00357 | 30 | 4 | 95 |
| PV00357 | 24 | 5 | 110 |
| Average | | | 114.25 |

### Example 4

### Determination of Functional Homologs by Reciprocal BLAST

A candidate sequence was considered a functional homolog of a reference sequence if the candidate and reference sequences encoded proteins having a similar function and/or activity. A process known as Reciprocal BLAST (Rivera et al., Proc. Natl. Acad. Sci. USA, 95:6239-6244 (1998)) was used to identify potential functional homolog sequences from databases consisting of all available public and proprietary peptide sequences, including NR from NCBI and peptide translations from Ceres clones.

Before starting a Reciprocal BLAST process, a specific reference polypeptide was searched against all peptides from its source species using BLAST in order to identify polypeptides having BLAST sequence identity of 80% or greater to the reference polypeptide and an alignment length of 85% or greater along the shorter sequence in the alignment. The reference polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the −postsw option. The BLAST sequence identity was calculated based on the alignment of the first BLAST HSP (High-scoring Segment Pairs) of the identified potential functional homolog sequence with a specific reference polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a reference polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original reference polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog.

Functional homologs were identified by manual inspection of potential functional homolog sequences. Representative functional homologs for SEQ ID NO:5 are shown in FIG. 1. Additional exemplary homologs are shown in the Sequence Listing.

### Example 5

### Determination of Functional Homologs by Hidden Markov Models

Hidden Markov Models (HMMs) were generated by the program HMMER 2.3.2. To generate each HMM, the default HMMER 2.3.2 program parameters, configured for global alignments, were used.

An HMM was generated using the sequences shown in FIG. 1 as input. These sequences were fitted to the model and a representative HMM bit score for each sequence is shown in the Sequence Listing. Additional sequences were fitted to the model, and representative HMM bit scores for any such additional sequences are shown in the Sequence Listing. The results indicate that these additional sequences are functional homologs of SEQ ID NO:5.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making switchgrass seed, said method comprising:
a) crossing a plurality of first switchgrass plants grown in pollinating proximity to a plurality of second switchgrass plants,
said first plants comprising a first exogenous nucleic acid, said first nucleic acid comprising a transcription factor activation sequence operably linked to a plant sterility sequence, wherein said first switchgrass plants are homozygous for said first exogenous nucleic acid,
said second plants comprising a second exogenous nucleic acid comprising a regulatory region operably linked to a coding sequence for a transcription factor that binds to said activation sequence, wherein said second switchgrass plants are homozygous for said second exogenous nucleic acid,
wherein said plurality of first switchgrass plants and/or said plurality of second switchgrass plants are clonally propagated plants; and
b) collecting F₁ seeds formed on said first and/or said second switchgrass plants, wherein F₁ switchgrass plants grown from said F₁ seeds express said plant sterility sequence and are sterile.

2. The method of claim 1, wherein said first switchgrass plants are clonally propagated plants and said second switchgrass plants are clonally propagated plants.

3. The method of claim 2, wherein said first clonally propagated switchgrass plants are octaploid plants and exhibit a self-compatibility percentage of less than 1.3%.

4. The method of claim 2, wherein said second clonally propagated switchgrass plants are tetraploid plants and exhibit an average self-compatibility percentage of less than 0.3%.

5. The method of claim 2, wherein said second clonally propagated switchgrass plants are octaploid plants and exhibit a self-compatibility percentage of less than 1.3%.

6. The method of claim 1, wherein said seeds are collected from both said first and said second switchgrass plants.

7. The method of claim 1, wherein said F₁ plants produce an average of less than 0.5 fertile seeds per plant.

8. The method of claim 7, wherein said F₁ plants are incapable of producing male and female gametes.

9. The method of claim 7, wherein said F₁ plants are incapable of producing male gametes.

10. The method of claim 7, wherein said F₁ plants are incapable of producing female gametes.

11. The method of claim 1, wherein the average crossability percentage between said first and said second switchgrass plants is from about 50% to about 95%.

12. The method of claim 11, wherein said first and said second switchgrass plants are tetraploid, are of the lowland ecotype, and have an average crossability percentage from about 80% to about 95%.

13. The method of claim 12, wherein said first and said second switchgrass plants have an average crossability percentage from about 86% to about 91%.

14. The method of claim 2, wherein said first switchgrass plants exhibit a uniform flowering time and said second switchgrass plants exhibit a non-uniform flowering time.

15. The method of claim 2, wherein said second switchgrass plants exhibit a compact inflorescence and said first switchgrass plants exhibit a diffuse inflorescence.

16. The method of claim 2, wherein said second switchgrass plants exhibit a uniform flowering time and said first switchgrass plants exhibit a non-uniform flowering time.

17. The method of claim 1, wherein said growing step comprises growing said switchgrass plants at a ratio of greater than 4:1 of said first switchgrass plants:second switchgrass plants.

18. The method of claim 1, wherein said growing step comprises growing said switchgrass plants at a ratio of greater than 4:1 of said second switchgrass plants:first switchgrass plants.

19. The method of claim 1, wherein said first and said second switchgrass plants are lowland type switchgrass plants.

20. The method of claim 1, wherein said first switchgrass plants further comprise an exogenous nucleic acid comprising said first transcription factor activation sequence operably linked to a second plant sterility sequence, and said first switchgrass plants exhibit homozygosity for said exogenous nucleic acid comprising said second plant sterility sequence.

21. The method of claim 1, wherein said first switchgrass plants further comprise an exogenous nucleic acid comprising a second transcription factor activation sequence operably linked to a second plant sterility sequence, and said first switchgrass plants exhibit homozygosity for said exogenous nucleic acid comprising said second plant sterility sequence; and wherein said second switchgrass plants further comprise an exogenous nucleic acid comprising a regulatory region operably linked to a coding sequence for a second transcription factor that binds to said second activation sequence, and exhibit homozygosity for said exogenous nucleic acid comprising said coding sequence for said second transcription factor.

22. The method of claim 1, wherein said first and/or said second switchgrass plants further comprise a transgene.

23. The method of claim 22, wherein said first and/or said second switchgrass plants exhibit homozygosity for said transgene.

24. The method of claim 1, wherein said plant sterility sequence encodes a polypeptide.

25. The method of claim 24, wherein an HMM bit score of the amino acid sequence of said polypeptide is greater than about 175, said HMM based on the amino acid sequences depicted in FIG. 1, and wherein said plant has decreased fertility as compared a control plant that does not comprise said nucleic acid.

26. The method of claim 24, wherein said polypeptide comprises an AP2 domain having at least 80% sequence identity to residues 134 to 185 of SEQ ID NO:5, a CMX-1 motif, and a CMX-2 motif.

27. The method of claim 26, wherein said polypeptide comprises an AP2 domain having at least 90% sequence identity to residues 134 to 185 of SEQ ID NO:5, a CMX-1 motif, and a CMX-2 motif.

28. The method of claim 26, wherein said polypeptide comprises an AP2 domain having at least 95% sequence identity to residues 134 to 185 of SEQ ID NO:5, a CMX-1 motif, and a CMX-2 motif.

29. The method of claim 26, wherein said polypeptide comprises an amino acid sequence with at least 85% sequence identity to a sequence selected from the group consisting of set forth in SEQ ID NOs:5, 6, 8, 10, 11, 13, 15, 17, 19, 21, 22, 24, 25, 26, 27, 28, 29, and 31.

30. The method of claim 1, wherein said plant sterility sequence comprises at least 50 contiguous nucleotides of any one of the nucleotide sequences set forth in SEQ ID NOs: 1, 2,3, and 32, and is transcribed into a transcription product.

31. The method of claim 1, wherein said transcription factor is a chimeric transcription factor comprising a binding domain selected from the group consisting of Hap1, LexA, Lac Operon, ArgR, AraC, PDR3, and LEU3 binding domain.

32. The method of claim 1, wherein said transcription factor is a chimeric transcription factor comprising an activation domain selected from the group consisting of VP16, C1 protein, ATMYB2, HAFL-1, ANT, ALM2, AvrXa10, Viviparous 1 (VP1), DOF, and RISBZ1 activation domain.

33. The method of claim 1, wherein said regulatory region is a broadly expressing promoter.

34. The method of claim 1, wherein said regulatory region is a photosynthetic tissue promoter.

35. The method of claim 1, wherein said plants grown from said F₁ seeds have a statistically significant increase in biomass in at least one growing season relative to control switchgrass plants that lack said first and said second exogenous nucleic acids.

36. F₁ switchgrass seeds made by the method set forth in claim 1.

37. A plurality of F₁ hybrid transgenic switchgrass seeds, said seeds made by a process comprising:
a) growing a plurality of first switchgrass plants in pollinating proximity to a plurality of second switchgrass plants,
said first plants comprising a first exogenous nucleic acid, said first nucleic acid comprising a transcription factor activation sequence operably linked to a plant sterility sequence,
said second plants comprising a second exogenous nucleic acid comprising a regulatory region operably linked to a coding sequence for a transcription factor that binds to said activation sequence,
wherein said plurality of first switchgrass plants and/or said plurality of second switchgrass plants are clonally propagated plants;
b) crossing said first switchgrass plants and said second switchgrass plants; and
c) collecting F₁ seeds formed on said first and/or said second switchgrass plants, wherein F₁ switchgrass plants grown from said F₁ seeds express said plant sterility sequence and are sterile.

38. The switchgrass seeds of claim 37, wherein said first switchgrass plants and said second switchgrass plants have crossability percentage of greater than about 65%.

39. A method for making switchgrass seed, said method comprising:
a) crossing a plurality of first switchgrass plants grown in pollinating proximity to a plurality of second switchgrass plants,
said first plants comprising a first exogenous nucleic acid, said first nucleic acid comprising a transcription factor activation sequence operably linked to a plant sterility sequence, said plant sterility sequence comprising at least 50 contiguous nucleotides of any one of the nucleotide sequences set forth in SEQ ID NOs: 1, 2, 3, or 32, wherein said first switchgrass plants are homozygous for said first exogenous nucleic acid,
said second plants comprising a second exogenous nucleic acid comprising a regulatory region operably linked to a coding sequence for a transcription factor that binds to said activation sequence, wherein said second switchgrass plants are homozygous for said second exogenous nucleic acid; and
b) collecting F₁ seeds formed on said first and/or said second switchgrass plants, wherein F₁ switchgrass plants grown from said F₁ seeds express said plant sterility sequence and are sterile.

40. A plurality of F₁ transgenic switchgrass seeds, said seeds comprising:
a) a first exogenous nucleic acid comprising a transcription upstream activation sequence (UAS) and a first promoter, wherein said UAS and said first promoter are operably linked to a first sequence encoding a first plant sterility sequence,
b) a second exogenous nucleic acid comprising said UAS and a second promoter, wherein said UAS and said second promoter are operably linked to a sequence encoding a second plant sterility sequence, wherein said first and said exogenous nucleic acids are different and affect a different developmental stage selected from the group consisting of i) spikelet meristem identity, ii) establishment of floral meristem identity, and iii) floral organ initiation, development, or function; and
c) a third exogenous nucleic acid comprising a third promoter operably linked to a transcription factor, wherein said transcription factor binds said UAS, wherein F₁ switchgrass plants grown from said F₁ seeds express said plant sterility sequences and are sterile.

41. A plurality of F₁ transgenic switchgrass seeds comprising:
a) a first exogenous nucleic acid comprising a first transcription upstream activation sequence (UAS) and a first promoter, wherein said first UAS and said first promoter are operably linked to a sequence encoding a first plant sterility sequence,
b) a second exogenous nucleic acid comprising a second UAS and a second promoter, wherein said second UAS and said second promoter are operably linked to a sequence encoding a second plant sterility sequence, wherein said first and said second exogenous nucleic acids are different and affect a different developmental stage selected from the group consisting of i) spikelet meristem identity, ii) establishment of floral meristem identity, and iii) floral organ initiation, development, or function;
c) a third exogenous nucleic acid comprising a third promoter operably linked to a transcription factor, wherein said transcription factor binds said first UAS; and
d) a fourth exogenous nucleic acid comprising a fourth promoter operably linked to a transcription factor, wherein said transcription factor binds said second UAS; wherein F₁ switchgrass plants grown from said F₁ seeds express said plant sterility sequences and are sterile.
